(19) United States
(12) Patent Application Publication
(10) Pub. No.: US 2010/0323953 A1
(43) Pub. Date: Dec. 23, 2010

(54) MACROCYCLIC HEPATITIS C PROTEASE INHIBITORS

(75) Inventors: David Alan Campbell, San Diego, CA (US); Michael E. Hepperle, Kreuzlingen (CH); David T. Winn, San Diego, CA (US); Juan Manuel Betancort, San Diego, CA (US)

Correspondence Address: SCHWEGMAN, LUNDBERG & WOESSNER, P.A., P.O. BOX 2938, MINNEAPOLIS, MN, 55402, US

(73) Assignee: Phenomix Corporation, San Diego, CA (US)

(21) Appl. No.: 12/526,018

(22) PCT Filed: Jan. 4, 2008
(86) PCT No.: PCT/US08/50208

§ 371 Date: Aug. 20, 2009

Related U.S. Application Data
(60) Provisional application No. 60/883,946, filed on Jan. 8, 2007.

(51) Int. Cl. A61K 38/12; C07K 5/12; A61P 31/14

(52) U.S. Cl. 514/4.3; 530/317

(57) ABSTRACT

The present invention provides novel macrocyclic compounds that mimic peptide substrates of the hepatitis C viral protease and inhibit the viral protease, more particularly as inhibitors of the NS3 serine protease from hepatitis C virus. Methods for synthesis of the compounds are also provided. The compounds find utility as antiviral agents directed at hepatitis C. The invention further provides methods of employing such inhibitors, alone or in combination with other therapeutic agents, to treat hepatitis C infection in a subject in need of such treatment.

### CROSS-REFERENCE TO RELATED APPLICATION

This claims the priority of U.S. Ser. No. 60/883,946, filed Jan. 8, 2007, which is incorporated herein by reference in its entirety.

### BACKGROUND

Hepatitis C virus (“HCV”) is the causative agent for hepatitis C, a chronic infection characterized by jaundice, fatigue, abdominal pain, loss of appetite, nausea, and darkening of the urine. HCV, belonging to the hepacivirus genus of the Flaviviriae family, is an enveloped, single-stranded positive-sense RNA-containing virus. The long-term effects of hepatitis C infection as a percentage of infected subjects include chronic infection (55-85%), chronic liver disease (70%), and death (1-5%). Furthermore, HCV is the leading indication for liver transplant. In chronic infection, there usually presents progressively worsening liver inflammation, which often leads to more severe disease states such as cirrhosis and hepatocellular carcinoma.

The HCV genome (Choo et al., Science 1989, 244, 359-362; Simmonds et al., Hepatology 1995, 21, 570-583) is a highly variable sequence exemplified by GenBank accession NC_{—}004102 as a 9646 base single-stranded RNA comprising the following constituents at the parenthetically indicated positions: 5′ NTR (i.e., non-transcribed region) (1-341); core protein (i.e., viral capsid protein involved in diverse processes including viral morphogenesis or regulation of host gene expression) (342-914); E1 protein (i.e., viral envelope) (915-1490); E2 protein (i.e., viral envelope) (1491-2579); p7 protein (2580-2768); NS2 protein (i.e., non-structural protein 2) (2769-3419); NS3 protease (3420-5312); NS4a protein (5313-5474); NS4b protein (5475-6257); NS5a protein (6258-7601); NS5b RNA-dependent RNA polymerase (7602-9372); and 3′ NTR (9375-9646). Additionally, a 17-kDalton −2/+1 frameshift protein, “protein F”, comprising the joining of positions (342-369) with (371-828) may provide functionality originally ascribed to the core protein.

The NS3 (i.e., non-structural protein 3) protein of HCV exhibits serine protease activity, the N-terminus of which is produced by the action of a NS2-NS3 metal-dependent protease, and the C-terminus of which is produced by auto-proteolysis. The HCV NS3 serine protease and its associated cofactor, NS4a, process all of the other non-structural viral proteins of HCV. Accordingly, the HCV NS3 protease is essential for viral replication.

Several compounds have been shown to inhibit the hepatitis C serine protease, but all of these have limitations in relation to the potency, stability, selectivity, toxicity, and/or pharmacodynamic properties. Such compounds have been disclosed, for example, in published U.S. Patent Application Nos. 2004/0266731, 2002/0032175, 2005/0137139, 2005/0119189, and 2004/9977600A1, and in published PCT patent applications WO 2005/037214 and WO 2005/035525.

### SUMMARY

The present invention provides macrocyclic compounds of Formula X that are adapted to inhibit the viral protease NS3 of the Hepatitis C Virus (HCV). The compounds of Formula X are adapted to bind to, and thus block the action of, an HCV-encoded protease enzyme that is required by the virus for the production of intact, mature, functional viral proteins from the viral polyprotein as translated from the viral RNA, and therefore for the formation of infectious particles, and ultimately for viral replication. The compounds of the invention are believed to act as mimics or analogs of the peptide domain immediately N-terminal of the substrate site where the viral protease cleaves its native substrate viral polyprotein.

Embodiments of the inventive compounds are analogs of peptides, comprising peptide (amide) bonds, inter alia, wherein a macrocyclic ring joins portions of the molecule, and wherein the group analogous to the C-terminus of a peptide is a carboxamide or analog thereof which can be unsubstituted or substituted with a range of substituents.

Accordingly, embodiments of the invention include a compound of Formula X:

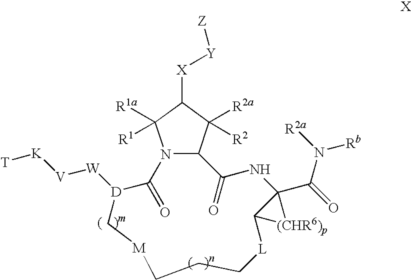

and stereoisomers, solvates, tautomers, prodrugs, salts, pharmaceutically acceptable salts, and mixtures thereof, wherein:

R^{a} and R^{b} at each occurrence are independently H, OR³, NR⁴R⁵, alkyl, alkenyl, aryl, aralkyl, aralkenyl, cycloalkyl, cycloalkylalkyl, cycloalkylalkenyl, cycloalkenyl, cycloalkenylalkyl, heterocyclyl, heterocyclylalkyl, heterocyclylalkenyl, heteroaryl, heteroarylalkyl, or heteroarylalkenyl, wherein any carbon atom can be substituted with J; or, R^{a} and R^{b}, together with a nitrogen atom to which they are bound, form a 3-8 membered heterocyclic ring which can be unsubstituted or substituted with 1-3 J, wherein the 3-8 membered heterocyclic ring can contain 1-3 additional heteroatoms selected from the group consisting of O, NR⁷, S, S(O), and S(O)₂, wherein the 3-8 membered heterocyclic ring can be fused with a substituted or unsubstituted, cycloalkyl, cycloalkenyl, aryl, heterocyclyl, or heteroaryl ring, or any combination thereof;

R¹, R^{1a}, R² and R^{2a} are independently H or alkyl, alkenyl, aryl, aralkyl, aralkenyl, cycloalkyl, cycloalkylalkyl, cycloalkylalkenyl, cycloalkenyl, cycloalkenylalkyl, heterocyclyl, heterocyclylalkyl, heterocyclylalkenyl, heteroaryl, heteroarylalkyl, or heteroarylalkenyl, wherein any carbon atom can be substituted with J;

R³, R⁴ and R⁵ are independently H or alkyl, alkenyl, aryl, aralkyl, aralkenyl, cycloalkyl, cycloalkylalkyl, cycloalkylalkenyl, cycloalkenyl, cycloalkenylalkyl, heterocyclyl, heterocyclylalkyl, heterocyclylalkenyl, heteroaryl, heteroarylalkyl, or heteroarylalkenyl, wherein any carbon atom can be substituted with J; or R⁴ and R⁵, together with a nitrogen atom to which they are bound, form a 3-8 membered heterocyclic ring which can be unsubstituted or substituted with 1-3 J, wherein the 3-8 membered heterocyclic ring can contain 1-3 additional heteroatoms selected from the group consisting of O, NR⁷, S, S(O), and S(O)₂, wherein the 3-8 membered heterocyclic ring can be fused with a cycloalkyl, cycloalkenyl, aryl, heterocyclyl, or heteroaryl ring, or any combination thereof;

D is CH₂, CH or N;

when D is CH₂, then W, V, K and T are absent;

when D is CH, then W is C(R⁶)₂, O, S, or NR⁷, and V, K, and T are as defined below;

when D is N then W, V and K are bonds, the bonds taken together forming a single bond, T is as defined below, such that T is bonded directly to D;

wherein R⁶ is independently at each occurrence hydrogen, alkyl, alkenyl, aryl, aralkyl, aralkenyl, cycloalkyl, cycloalkylalkyl, cycloalkylalkenyl, cycloalkenyl, cycloalkenylalkyl, heterocyclyl, heterocyclylalkyl, heterocyclylalkenyl, heteroaryl, heteroarylalkyl, or heteroarylalkenyl, wherein any carbon atom can be substituted with J; or wherein two R⁶ groups together with a carbon atom to which they are bond form a 3-8 membered cycloalkyl, which can be unsubstituted or substituted with 1-3 J, wherein the 3-8 membered cycloalkyl can contain 1-3 heteroatoms selected from the group consisting of O, NR⁷, S, S(O), and S(O)₂, wherein the 3-8 membered cycloalkyl can be fused with a cycloalkyl, cycloalkenyl, aryl, heterocyclyl, or heteroaryl ring, or any combination thereof;

R⁷ is independently at each occurrence hydrogen, alkyl, alkenyl, aryl, aralkyl, aralkenyl, cycloalkyl, cycloalkylalkyl, cycloalkylalkenyl, cycloalkenyl, cycloalkenylalkyl, heterocyclyl, heterocyclylalkyl, heterocyclylalkenyl, heteroaryl, heteroarylalkyl, or heteroarylalkenyl, wherein any carbon atom can be substituted with J, or aralkanoyl, heteroaralkanoyl, C(O)R⁸, SO₂R⁸ or carboxamido, wherein any aralkanoyl or heteroaralkanoyl is substituted with 0-3 J groups;

R⁸ is alkyl, alkenyl, aryl, aralkyl, aralkenyl, cycloalkyl, cycloalkylalkyl, cycloalkylalkenyl, cycloalkenyl, cycloalkenylalkyl, heterocyclyl, heterocyclylalkyl, heterocyclylalkenyl, heteroaryl, heteroarylalkyl, or heteroarylalkenyl, wherein any carbon atom can be substituted with J;

m is 1, 2, 3 or 4;

n is 0, 1, 2, 3 or 4;

p is 1, 2, 3, or 4;

M is O, S, S(O), S(O)₂, C(R⁶)₂ or N(R⁷);

J is halogen, R′, OR′, CN, CF₃, OCF₃, O, S, C(O), S(O), methylenedioxy, ethylenedioxy, (CH₂)₀₋₂N(R′)₂, (CH₂)₀₋₂SR′, (CH₂)₀₋₂S(O)R′, (CH₂)₀₋₂S(O)₂R′, (CH₂)₀₋₂S(O)₂N(R′)₂, (CH₂)₀₋₂SO₃R′, (CH₂)₀₋₂C(O)R′, (CH₂)₀₋₂C(O)C(O)R′, (CH₂)₀₋₂C(O)CH₂C(O)R′, (CH₂)₀₋₂C(S)R′, (CH₂)₀₋₂C(O)OR′, (CH₂)₀₋₂C(O)R′, (CH₂)₀₋₂C(O)N(R′)₂, (CH₂)₀₋₂C(O)N(R′)₂, (CH₂)₀₋₂C(S)N(R′)₂, (CH₂)₀₋₂NH—C(O)R′, (CH₂)₀₋₂N(R′)N(R′)C(O)R′, (CH₂)₀₋₂N(R′)N(R′)C(O)R′, (CH₂)₀₋₂N(R′)N(R)CON(R′)₂, (CH₂)₀₋₂N(R′)SO₂R′, (CH₂)₀₋₂N(R′)SO₂N(R′)₂, (CH₂)₀₋₂N(R′)C(O)OR′, (CH₂)₀₋₂N(R′)C(O)R′, (CH₂)₀₋₂N(R′)C(S)R′, (CH₂)₀₋₂N(R′)C(O)N(R′)₂, (CH₂)₀₋₂N(R′)C(S)N(R′)₂, (CH₂)₀₋₂N(COR′)COR′, (CH₂)₀₋₂N(OR′)R′, (CH₂)₀₋₂C(═NH)N(R′)₂, (CH₂)₀₋₂C(O)N(OR′)R′, or (CH₂)₀₋₂C(═NOR′)R′; wherein,

each R′ is independently at each occurrence hydrogen, (C₁-C₁₂)-alkyl, (C₂-C₁₂)-alkenyl, (C₂-C₁₂)-alkynyl, (C₃-C₁₀)-cycloalkyl, (C₃-C₁₀)-cycloalkenyl, [(C₃-C₁₀)cycloalkyl or (C₃-C₁₀)-cycloalkenyl]-[(C₁-C₁₂)-alkyl or (C₂-C₁₂)-alkenyl or (C₂-C₁₂)-alkynyl], (C₆-C₁₀)-aryl, (C₆-C₁₀-aryl-[(C₁-C₁₂)-alkyl or (C₂-C₁₂)-alkenyl or (C₂-C₁₂)-alkynyl], (C₃-C₁₀)-heterocyclyl, (C₃-C₁₀)-heterocyclyl-[(C₁-C₁₂)-alkyl or (C₂-C₁₂)-alkenyl or (C₂-C₁₂)-alkynyl], (C₅-C₁₀)-heteroaryl, or (C₅-C₁₀)-heteroaryl-[(C₁-C₁₂)-alkyl or (C₂-C₁₂)-alkenyl or (C₂-C₁₂)-alkynyl], wherein R′ is substituted with 0-3 substituents selected independently from J;

or, when two R′ are bound to a nitrogen atom or to two adjacent nitrogen atoms, the two R′ groups together with the nitrogen atom or atoms to which they are bound can form a 3- to 8-membered monocyclic heterocyclic ring, or an 8- to 20-membered, bicyclic or tricyclic, heterocyclic ring system, wherein any ring or ring system can further contain 1-3 additional heteroatoms selected from the group consisting of N, NR⁷, O, S, S(O) and S(O)₂, wherein each ring is substituted with 0-3 substituents selected independently from J;

wherein, in any bicyclic or tricyclic ring system, each ring is linearly fused, bridged, or spirocyclic, wherein each ring is either aromatic or nonaromatic, wherein each ring can be fused to a (C₆-C₁₀)aryl, (C₅-C₁₀) heteroaryl, (C₃-C₁₀)cycloalkyl or (C₃-C₁₀) heterocyclyl;

L is O, S, C₂, C₂H₂ or C₂H₄;

V is a bond, C(R¹⁰)₂, C(O), S(O), or S(O)₂;

K is a bond, O, S, C(O), S(O), S(O)₂, S(O)(NR⁷), or N(R⁷);

provided that when V and K are both bonds, they form together a single bond;

R¹⁰ is independently at each occurrence hydrogen, alkyl, cycloalkyl, cycloalkylalkyl, aryl, aralkyl, heterocyclyl, heterocyclylalkyl, heteroaryl or heteroarylalkyl; or, two R¹⁰ groups together with a carbon atom to which they are bound form a 3-8 membered cycloalkyl, which can be unsubstituted or substituted with 1-3 J, wherein the 3-8 membered cycloalkyl can contain 1-3 heteroatoms selected from the group consisting of O, NR⁷, S, S(O), and S(O)₂, wherein the 3-8 membered cycloalkyl can be fused with a cycloalkyl, cycloalkenyl, aryl, heterocyclyl, or heteroaryl ring, or any combination thereof;

T is R¹¹, alkyl-R¹¹, alkenyl-R¹¹, alkynyl-R¹¹, OR¹¹, N(R¹¹)₂, C(O)R¹¹, or C(═NOalkyl)R¹¹;

R¹¹ is independently at each occurrence hydrogen, alkyl, aryl, aralkyl, alkoxy, amino, alkylamino, dialkylamino, cycloalkyl, cycloalkenyl, [cycloalkyl or cycloalkenyl]-[alkyl or alkenyl], heterocyclyl, heterocyclylalkyl, heterocyclylalkenyl, heteroaryl, heteroarylalkyl, or heteroarylalkenyl, wherein any R¹¹ except hydrogen is substituted with 0-3 J groups, or a first R¹¹ and a second R¹¹ together with a nitrogen atom to which they are bound form a mono- or bicyclic ring system substituted with 0-3 J groups and can contain 1-3 additional heteroatoms selected from the group consisting of O, NR⁷, S, S(O), and S(O)₂; and

when W is C(R⁶)₂, a bond, or absent;

X is a bond, O, S, CH(R⁶) or N(R⁷);

Y is a bond, CH(R⁶), C(O), C(O)C(O), S(O), S(O)₂, or S(O)(NR⁷);

provided that when both X and Y are bonds, they together form a single bond;

Z is:
a) hydrogen, alkyl, aryl, aralkyl, cycloalkyl, cycloalkylalkyl, heterocyclyl, heterocyclylalkyl, heteroaryl, heteroarylalkyl, OR⁹, or N(R⁹)₂, wherein any carbon atom is unsubstituted or is substituted with J, and wherein R⁹ is independently at each occurrence hydrogen, alkyl, alkenyl, aryl, aralkyl, aralkenyl, [cycloalkyl or cycloalkenyl]-[alkyl or alkenyl], heterocyclyl, heterocyclylalkyl, heterocyclylalkenyl, heteroaryl, or heteroarylalkyl, or two R⁹ groups together with a nitrogen atom to which they are bound can form together with the nitrogen atom a 5-11 membered mono- or bicyclic heterocyclic ring system substituted with 0-3 J groups and further including 0-3 additional heteroatoms selected from the group consisting of O, NR⁷, S, S(O), and S(O)₂;

or
b) a substituted aryl or heteroaryl group; wherein any aryl or heteroaryl is substituted with 1-3 J groups;

or
c) a group of the formula:

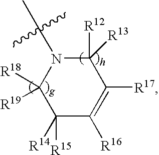

wherein
R¹², R¹³, R¹⁴, R¹⁵, R¹⁸ and R¹⁹ are independently hydrogen, fluorine, or a substituted or unsubstituted alkyl, cycloalkyl, cycloalkenyl, [cycloalkyl or cycloalkenyl]-[alkyl or alkenyl], aryl, aralkyl, aralkenyl, heterocyclyl, heterocyclylalkyl, heterocyclylalkenyl, heteroaryl, heteroarylalkyl, or heteroarylalkenyl group; or R¹² and R¹³ or R¹⁴ and R¹⁵ or R¹⁸ and R¹⁹, together with a carbon atom to which they are attached, form a C₃₋₆ cycloalkyl group;
R¹⁶ and R¹⁷ are independently hydrogen, fluorine, or a substituted or unsubstituted alkyl, cycloalkyl, cycloalkenyl, [cycloalkyl or cycloalkenyl]-[alkyl or alkenyl], aryl, aralkyl, aralkenyl, heterocyclyl, heterocyclylalkyl, heterocyclylalkenyl, heteroaryl, heteroarylalkyl, or heteroarylalkenyl group; or R¹⁶ and R¹⁷ together with the atoms to which they are attached form a fused substituted or unsubstituted aryl or heteroaryl group;
g is 0-1; and
h is 0-2;

or
d) a group of the formula:

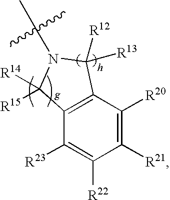

wherein
g is 0-2; and
h is 0-2;
R¹², R¹³, R¹⁴, and R¹⁵ are independently at each occurrence hydrogen, fluorine, or a substituted or unsubstituted alkyl, cycloalkyl, cycloalkenyl, [cycloalkyl or cycloalkenyl]-[alkyl or alkenyl], aryl, aralkyl, aralkenyl, heterocyclyl, heterocyclylalkyl, heterocyclylalkenyl, heteroaryl, heteroarylalkyl, or heteroarylalkenyl group; or R¹² and R¹³ or R¹⁴ and R¹⁵, together with a carbon atom to which they are attached, form a C₃₋₆ cycloalkyl group;
R²⁰, R²¹, R²², R²³ are independently H, F, Cl, Br, I, CN, CF₃, OCF₃, OR²⁴, (CH₂)OR²⁴, O(CH₂)_{q}OR²⁴, NR²⁵R²⁶, (CH₂)_{q}NR²⁵R²⁶, O(CH₂)_{q}NR²⁵R²⁶, SR²⁴, (CH₂)_{q}SR²⁴, O(CH₂)_{q}SR²⁴, C(O)R²⁴, (CH₂)_{q}C(O)R²⁴, O(CH₂)_{q}C(O)R²⁴, C(O)OR²⁴, (CH₂)_{q}C(O)OR²⁴, O(CH₂)_{q}C(O)OR²⁴, NR²⁷C(O)R²⁴, (CH₂)_{q}NR²⁷C(O)R²⁴, O(CH₂)_{q}NR²⁷(O)R²⁴, C(O)NR²⁵R²⁶, (CH₂)_{q}C(O)NR²⁵R²⁶, O(CH₂)_{q}C(O)NR²⁵R²⁶, NR²⁷C(O)NR²⁵R²⁶, (CH₂)_{q}NR²⁷C(O)NR²⁵R²⁶, O(CH₂)_{q}NR²⁷C(O)NR²⁵R²⁶, OC(O)NR²⁵R²⁶, (CH₂)_{q}OC(O)NR²⁵R²⁶, O(CH₂)_{q}OC(O)NR²⁵R²⁶, NR²⁷C(O)OR²⁴, (CH₂)_{q}NR²⁷C(O)OR²⁴, O(CH₂)_{q}NR²⁷C(O)OR²⁴, NR²⁷SO₂R²⁴, (CH₂)_{q}NR²⁷SO₂R²⁴, O(CH₂)_{q}NR²⁷SO₂R²⁴, SO₂NR²⁵R²⁶, (CH₂)_{q}SO₂NR²⁵R²⁶, or O(CH₂)_{q}SO₂NR²⁵R²⁶, or a substituted or unsubstituted alkyl, cycloalkyl, cycloalkenyl, [cycloalkyl or cycloalkenyl]-[alkyl or alkenyl], aryl, aralkyl, aralkenyl, heterocyclyl, heterocyclylalkyl, heterocyclylalkenyl, heteroaryl, heteroarylalkyl, or heteroarylalkenyl group,
q is 1, 2, 3, 4, 5, or 6; and
each R²⁴, R²⁵, R²⁶, and R²⁷ is independently hydrogen, or a substituted or unsubstituted alkyl, cycloalkyl, cycloalkenyl, [cycloalkyl or cycloalkenyl]-[alkyl or alkenyl], aryl, aralkyl, arylalkenyl, heterocyclyl, heterocyclylalkyl, heterocyclylalkenyl, heteroaryl, heteroarylalkyl, or heteroarylalkenyl group; or R²⁵ and R²⁶ together with a nitrogen atom to which they are attached form a 3-7 membered heterocyclic ring substituted with 0-3 J groups, that further comprises 0-3 additional heteroatoms selected from the groups consisting of O, NR⁷, S, S(O), and S(O)₂;

or
e) a group of the formula

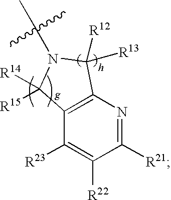

wherein
g is 0-2; and
h is 0-2;
R¹², R¹³, R¹⁴, R¹⁵, R²¹, R²² and R²³ are as defined in (d);

or
f) a group of the formula

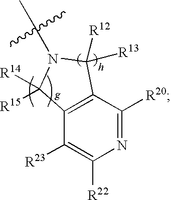

wherein
g is 0-2; and
h is 0-2;
R¹², R¹³, R¹⁴, R¹⁵, R²⁰, R²² and R²³ (are as defined in (d); and

wherein a wavy line signifies a point of attachment;

and,

when W is NR⁷, O or S:

X is O, CH₂, or NR';

Y is C(R⁶)₂ or absent; and

Z is a substituted alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, alkoxy, aryloxy, alkylthio, arylthio, alkylamino, arylamino, heteroaryl, or heteroarylalkyl; wherein any alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, alkoxy, aryloxy, alkylthio, arylthio, alkylamino, arylamino, heteroaryl, or heteroarylalkyl is substituted with 1-3 J groups, provided that K and V are both bonds, taken together forming a single bond such that T is bonded directly to W, T is not C(O)R¹¹;

or,

X is O;

Y is C(O);

Z is
aa) a group of the formula

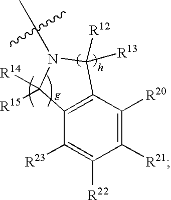

wherein
g is 0-2; and
h is 0-2;
R¹², R¹³, R¹⁴, and R¹⁵ are independently at each occurrence hydrogen, fluorine, or a substituted or unsubstituted alkyl, cycloalkyl, cycloalkenyl, [cycloalkyl or cycloalkenyl]-[alkyl or alkenyl], aryl, aralkyl, aralkenyl, heterocyclyl, heterocyclylalkyl, heterocyclylalkenyl, heteroaryl, heteroarylalkyl, or heteroarylalkenyl group; or R¹² and R¹³ or R¹⁴ and R¹⁵, together with a carbon atom to which they are attached, form a C₃₋₆ cycloalkyl group;
R²⁰, R²¹, R²², R²³ are independently H, F, Cl, Br, I, CN, CF₃, OCF₃, OR²⁴, (CH₂)_{q}OR²⁴, O(CH₂)_{q}OR²⁴, NR²⁵R²⁶, (CH₂)_{q}NR²⁵R²⁶, O(CH₂)_{q}NR²⁵R²⁶, SR²⁴, (CH₂)_{q}SR²⁴, O(CH₂)_{q}SR²⁴, C(O)R²⁴, (CH₂)_{q}C(O)R²⁴, O(CH₂)_{q}C(O)R²⁴, C(O)OR²⁴, (CH₂)_{q}C(O)OR²⁴, O(CH₂)_{q}C(O)OR²⁴, NR²⁷C(O)R²⁴, (CH₂)_{q}NR²⁷C(O)R²⁴, O(CH₂)_{q}NR²⁷C(O)R²⁴, C(O)NR²⁵R²⁶, (CH₂)_{q}C(O)NR²⁵R²⁶, O(CH₂)_{q}(C(O)NR²⁵R²⁶, NR²⁷C(O)NR²⁵R²⁶, (CH₂)_{q}NR²⁷C(O)NR²⁵R²⁶, O(CH₂)_{q}NR²⁷C(O)NR²⁵R²⁶, OC(O)NR²⁵R²⁶, (CH₂)_{q}OC(O)NR²⁵R²⁶, O(CH₂)_{q}OC(O)NR²⁵R²⁶, NR²⁷C(O)OR²⁴, (CH₂)_{q}NR²⁷C(O)OR²⁴, O(CH₂)_{q}NR²⁷C(O)OR²⁴, NR²⁷SO₂R²⁴, (CH₂)_{q}NR²⁷SO₂R²⁴, O(CH₂)_{q}NR²⁷SO₂R²⁴, SO₂NR²⁵R²⁶, (CH₂)_{q}SO₂NR²⁵R²⁶, or O(CH₂)_{q}SO₂NR²⁵R²⁶, or a substituted or unsubstituted alkyl, cycloalkyl, cycloalkenyl, [cycloalkyl or cycloalkenyl]-[alkyl or alkenyl], aryl, aralkyl, aralkenyl, heterocyclyl, heterocyclylalkyl, heterocyclylalkenyl, heteroaryl, heteroarylalkyl, or heteroarylalkenyl group,
q is 1, 2, 3, 4, 5, or 6; and
each R²⁴, R²⁵, R²⁶, and R²⁷ is independently hydrogen, or a substituted or unsubstituted alkyl, cycloalkyl, cycloalkenyl, [cycloalkyl or cycloalkenyl]-[alkyl or alkenyl], aryl, aralkyl, arylalkenyl, heterocyclyl, heterocyclylalkyl, heterocyclylalkenyl, heteroaryl, heteroarylalkyl, or heteroarylalkenyl group; or R²⁵ and R²⁶ together with a nitrogen atom to which they are attached form a 3-7 membered heterocyclic ring substituted with 0-3 J groups, that further comprises 0-3 additional heteroatoms selected from the groups consisting of O, NR⁷, S, S(O), and S(O)₂;

or
bb) a group of the formula

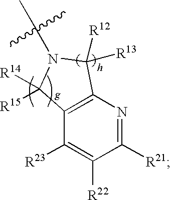

wherein
   g is 0-2; and
   h is 0-2;
   R¹², R¹³, R¹⁴, R¹⁵, R²⁰, R²² and R²³ are as defined in (aa);

or
cc) a group of the formula

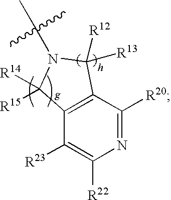

wherein
   g is 0-2; and
   h is 0-2;
   R¹², R¹³, R¹⁴, R¹⁵, R²⁰, R²² and R²³ are as defined in (c);

wherein a wavy line signifies a point of attachment.

The invention further provides a method for synthesis of a compound of Formula X.

The invention further provides a pharmaceutical composition comprising a compound of Formula X and a suitable excipient.

The invention further provides a pharmaceutical combination comprising a compound of Formula X in a therapeutically effective amount and a second medicament in a therapeutically effective amount. The pharmaceutical combination of the invention may be formulated as a pharmaceutical composition of the invention.

The present invention further provides a method of treatment of a HCV infection in a patient in need thereof, or in a patient when inhibition of an HCV viral protease is medically indicated, comprising administering a therapeutically effective amount of a compound of Formula Ito the patient, or a pharmaceutical combination to the patient.

### DETAILED DESCRIPTION OF THE INVENTION

### Definitions

The terms “HCV NS3 serine protease”, “HCV NS3 protease”, “NS3 serine protease”, and “NS3 protease” denote all active forms of the serine protease encoded by the NS3 region of the hepatitis C virus, including all combinations thereof with other proteins in either covalent or noncovalent association. For example, other proteins in this context include without limitation the protein encoded by the NS4a region of the hepatitis C virus. Accordingly, the terms “NS3/4a” and “NS3/4a protease” denote the NS3 protease in combination with the HCV NS4a protein.

The term “other type(s) of therapeutic agents” as employed herein refers to one or more antiviral agents (other than HCV NS3 serine protease inhibitors of the invention).

“Subject” as used herein, includes mammals such as humans, non-human primates, rats, mice, dogs, cats, horses, cows and pigs.

The term “treatment” is defined as the management and care of a patient for the purpose of combating the disease, condition, or disorder and includes administering a compound of the present invention to prevent the onset of the symptoms or complications, or alleviating the symptoms or complications, or eliminating the disease, condition, or disorder.

“Treating” within the context of the instant invention means an alleviation of symptoms associated with a disorder or disease, or inhibition of further progression or worsening of those symptoms, or prevention or prophylaxis of the disease or disorder. Thus, treating a hepatitis C viral infection includes slowing, halting or reversing the growth of the virus and/or the control, alleviation or prevention of symptoms of the infection. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result by inhibition of HCV NS3 serine protease activity. A therapeutically effective amount is also one in which any toxic or detrimental effects of compounds of the invention are outweighed by the therapeutically beneficial effects. For example, in the context of treating HCV infection, a therapeutically effective amount of a HCV NS3 serine protease inhibitor of the invention is an amount sufficient to control HCV viral infection.

All chiral, diastereomeric, racemic forms of a structure are intended, unless the specific stereochemistry or isomeric form is specifically indicated. Compounds used in the present invention include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these are all within the scope of the invention.

The term “amino protecting group” or “N-protected” as used herein refers to those groups intended to protect an amino group against undesirable reactions during synthetic procedures and which can later be removed to reveal the amine. Commonly used amino protecting groups are disclosed in Protective Groups in Organic Synthesis, Greene, T. W.; Wuts, P. G. M., John Wiley & Sons, New York, N.Y., (3rd Edition, 1999). Amino protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxy-carbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Amine protecting groups also include cyclic amino protecting groups such as phthaloyl and dithiosuccinimidyl, which incorporate the amino nitrogen into a heterocycle. Typically, amino protecting groups include formyl, acetyl, benzoyl, pivaloyl, t-butylacetyl, phenylsulfonyl, Alloc, Teoc, benzyl, Fmoc, Boc and Cbz. It is well within the skill of the ordinary artisan to select and use the appropriate amino protecting group for the synthetic task at hand.

In general, “substituted” refers to an organic group as defined herein in which one or more bonds to a hydrogen atom contained therein are replaced by a bond to non-hydrogen or non-carbon atoms such as, but not limited to, a halogen (i.e., F, Cl, Br, and I); an oxygen atom in groups such as hydroxyl groups, alkoxy groups, aryloxy groups, aralkyloxy groups; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, N-oxides, hydrazides, azides, and enamines; and other heteroatoms in various other groups. Substituted alkyl, alkenyl, alkynyl, cycloalkyl, and cycloalkenyl groups as well as other substituted groups also include groups in which one or more bonds to a carbon(s) or hydrogen(s) atom are replaced by one or more bonds, including double or triple bonds, to a heteroatom such as, but not limited to, oxygen in carbonyl (oxo), carboxyl, ester, amide, imide, urethane, and urea groups; and nitrogen in imines, hydroxyimines, oximes, hydrazones, amidines, guanidines, and nitriles.

When a group is defined as being substituted, it is understood that the substitution is “chemically feasible”, that is, that the substitution can be made without violating any of the well-known rules of chemical bonding known to those of skill in the art. For example, if a particular substitution of a chemical group would result in the presence of a pentavalent carbon atom in the structure, it is understood that the particular substitution of the chemical group would not be contemplated.

When a substituent is expressed in a combinatorial manner, as in a Claim, for example “[cycloalkyl or cycloalkenyl]-[alkyl or alkenyl]”, what is meant is all possible combinations of the options in the first alternative and the options in the second alternative; thus the above example includes cycloalkylalkyl, cycloalkylalkenyl, cycloalkenylalkyl, and cycloalkenylalkenyl.

The term “heteroatoms” as used herein refers to non-carbon and non-hydrogen atoms, and is not otherwise limited. Typical heteroatoms are N, O, and S. When sulfur (S) is referred to, it is understood that the sulfur can be in any of the oxidation states in which it is found, thus including sulfoxides (R—S(O)—R′) and sulfones (R—S(O)₂—R′), unless the oxidation state is specified; thus, the term “sulfone” encompasses only the sulfone form of sulfur; the term “sulfide” encompasses only the sulfide (R—S—R′) form of sulfur. When the phrases such as “heteroatoms selected from the group consisting of O, NH, NR′ and S,” or “[variable] is O, S . . . ” are used, they are understood to encompass all of the sulfide, sulfoxide and sulfone oxidation states of sulfur.

Substituted ring groups such as substituted aryl, heterocyclyl and heteroaryl groups also include rings and fused ring systems in which a bond to a hydrogen atom is replaced with a bond to a carbon atom. Therefore, substituted aryl, heterocyclyl and heteroaryl groups may also be substituted with alkyl, alkenyl, and alkynyl groups as defined herein.

Alkyl groups include straight chain and branched alkyl groups and cycloalkyl groups having from 1 to about 20 carbon atoms, and typically from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. Representative substituted alkyl groups may be substituted one or more times with any of the groups listed above, for example, amino, hydroxy, cyano, carboxy, nitro, thio, alkoxy, and halogen groups.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which may be substituted with, for example, amino, hydroxy, cyano, carboxy, nitro, thio, alkoxy, and halogen groups. The term “cycloalkenyl” alone or in combination denotes a cyclic alkenyl group.

The terms “carbocyclic” and “carbocycle” denote a ring structure wherein the atoms of the ring are carbon. In some embodiments, the carbocycle has 3 to 8 ring members, whereas in other embodiments the number of ring carbon atoms is 4, 5, 6, or 7. Unless specifically indicated to the contrary, the carbocyclic ring may be substituted with as many as N−1 substituents wherein N is the size of the carbocyclic ring with for example, amino, hydroxy, cyano, carboxy, nitro, thio, alkoxy, and halogen groups.

A “macrocyclic” molecule, or a “macrocycle,” as the term is used herein, refers to a cyclic organic structure wherein a ring has more than about 7 members. Thus, a macrocyclic ring can have 8, 9, 10, 11, 12, 13, 14, or more, members. The atoms making up this ring can be carbon, which can also include heteroatoms such as O, N, and S (in its various oxidation states, i.e., S, SO, or SO₂). Therefore, a macrocycle can include in the macrocyclic ring carbon chains and peptide (amide) bonds, as well as other moieties such as ethers, sulfides, sulfoxides, sulfones, amines, hydrazines, and the like.

(Cycloalkyl)alkyl groups, also denoted cycloalkylalkyl, are alkyl groups as defined above in which a hydrogen or carbon bond of the alkyl group is replaced with a bond to a cycloalkyl group as defined above.

Alkenyl groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

Cycloalkenyl groups include cycloalkyl groups having at least one double bond between 2 carbons. Thus for example, cycloalkenyl groups include but are not limited to cyclohexenyl, cyclopentenyl, and cyclohexadienyl groups.

(Cycloalkenyl)alkyl groups are alkyl groups as defined above in which a hydrogen or carbon bond of the alkyl group is replaced with a bond to a cycloalkenyl group as defined above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

Aryl groups are cyclic aromatic hydrocarbons that do not contain heteroatoms. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain 6-14 carbons in the ring portions of the groups. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halogen groups, bonded to one of the ring members. Rather, groups such as tolyl are referred to as substituted aryl groups. Representative substituted aryl groups may be mono-substituted or substituted more than once, such as, but not limited to, 2-, 3-, 4-, 5-, or 6-substituted phenyl or naphthyl groups, which may be substituted with groups such as those listed above.

Aralkyl groups are alkyl groups as defined above in which a hydrogen or carbon bond of an alkyl group is replaced with a bond to an aryl group as defined above. Representative aralkyl groups include benzyl and phenylethyl groups and fused (cycloalkylaryl)alkyl groups such as 4-ethyl-indanyl. Aralkenyl group are alkenyl groups as defined above in which a hydrogen or carbon bond of an alkyl group is replaced with a bond to an aryl group as defined above.

Heterocyclyl groups include aromatic and non-aromatic ring compounds containing 3 or more ring members, of which, one or more is a heteroatom such as, but not limited to, N, O, and S. In some embodiments, heterocyclyl groups include 3 to 20 ring members, whereas other such groups have 3 to 15 ring members. The phrase “heterocyclyl group” includes fused ring species including those comprising fused aromatic and non-aromatic groups. The phrase also includes polycyclic ring systems containing a heteroatom such as, but not limited to, quinuclidyl. However, the phrase does not include heterocyclyl groups that have other groups, such as alkyl or halogen groups, bonded to one of the ring members. Rather, these are referred to as “substituted heterocyclyl groups”. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups may be mono-substituted or substituted more than once, such as, but not limited to, piperidinyl or quinolinyl groups, which are 2-, 3-, 4-, 5-, or 6-substituted, or disubstituted with groups such as those listed above.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Although the phrase “heteroaryl groups” includes fused ring compounds such as indolyl and 2,3-dihydro indolyl, the phrase does not include heteroaryl groups that have other groups bonded to one of the ring members, such as alkyl groups. Rather, heteroaryl groups with such substitution are referred to as “substituted heteroaryl groups”. Representative substituted heteroaryl groups may be substituted one or more times with groups such as those listed above.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl-1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[bf]azepine (10,11-dihydro-5H-dibenz[b,f]azepine-1-yl, 10,11-dihydro-5H-dibenz[bf]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

Heterocyclylalkyl groups are alkyl groups as defined above in which a hydrogen or carbon bond of an alkyl group is replaced with a bond to a heterocyclyl group as defined above. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

Heteroarylalkyl groups are alkyl groups as defined above in which a hydrogen or carbon bond of an alkyl group is replaced with a bond to a heteroaryl group as defined above.

The term “alkoxy” refers to an oxygen atom connected to an alkyl group as defined above. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like.

The terms “aryloxy” and “arylalkoxy” refer to, respectively, an aryl group bonded to an oxygen atom and an aralkyl group bonded to the oxygen atom at the alkyl. Examples include but are not limited to phenoxy, naphthyloxy, and benzyloxy.

The term “amine” (or “amino”) includes primary, secondary, and tertiary amines having, e.g., the formula —NR₂. Amines include but are not limited to —NH₂, alkylamines, dialkylamines, arylamines, alkylarylamines, diarylamines, aralkylamines, heterocyclylamines and the like.

The term “amide” (or “amido”) includes C- and N-amide groups, i.e., —C(O)NR₂, and —NRC(O)R groups, respectively. Amide groups therefore include but are not limited to carbamoyl groups (—C(O)NH₂) and formamide groups (—NHC(O)H).

The term “urethane” (or “carbamyl”) includes N- and O-urethane groups, i.e., —NRC(O)OR and —OC(O)NR₂ groups, respectively.

The term “sulfonamide” (or “sulfonamido”) includes S- and N-sulfonamide groups, i.e., —SO₂NR₂ and —NRSO₂R groups, respectively. Sulfonamide groups therefore include but are not limited to sulfamoyl groups (—SO₂NH₂). An organosulfur structure represented by the formula —S(O)(NR)— is understood to refer to a sulfoximine, wherein both the oxygen and the nitrogen atoms are bonded to the sulfur atom, which is also bonded to two carbon atoms.

The term “amidine” or “amidino” includes groups of the formula —C(NR)NR₂. Typically, an amidino group is —C(NH)NH₂.

The term “guanidine” or “guanidino” includes groups of the formula —NRC(NR)NR₂. Typically, a guanidino group is —NHC(NH)NH₂.

In addition, where features or aspects of the invention are described in terms of Markush groups, those skilled in the art will recognize that the invention is also thereby described in terms of any individual member or subgroup of members of the Markush group. For example, if A is described as selected from the group consisting of bromine, chlorine, and iodine, claims for A being bromine and claims for A being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if A is described as selected from the group consisting of bromine, chlorine, and iodine, and B is described as selected from the group consisting of methyl, ethyl, and propyl, claims for A being bromine and B being methyl are fully described.

Without wishing to be bound by theory, the standard nomenclature of Schechter & Berger (Biochem. Biophys. Res. Comm., 1967, 27, 157-162) regarding the identification of residues in the polypeptide substrate of serine proteases will be employed herein unless other indicia of identification are specifically provided. Within the nomenclature of Schechter & Berger, the residues of the substrate, in the direction from the N-terminal toward the C-terminal, are labeled (Pi, . . . , P3, P2, P1, P1′, P2′, Pr′ . . . , Pj), wherein cleavage is catalyzed between P1 and P1′. Within the context of this nomenclature, compounds of Formulas X can be considered as mimics of at least the tripeptide P3-Pro-P1, wherein the analog of P1, as a moiety of the macrocyclic structure, is:

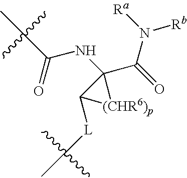

wherein R^{a}, R^{b}, L and p are as defined below, wherein the two wavy lines signify two respective points of attachment, and wherein the two points of attachment are ultimately connected to each other via a macrocyclic ring. The inventive compounds include an unsubstituted or a substituted carboxamide moiety or analog thereof at the carboxy terminus of the P1 analog.

The present invention provides a compound of Formula X:

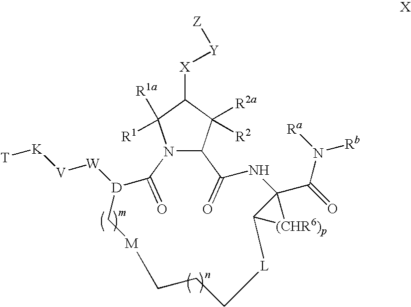

and stereoisomers, solvates, tautomers, prodrugs, salts, pharmaceutically acceptable salts, and mixtures thereof, wherein: R^{a}, R^{b}, R¹, R^{1a}, R², R^{2a}, R³, R⁴, R⁵, D, R⁶, R⁷, R⁸, R′, J, L, M, W, V, K, T, X, Y, Z, p, m and n, and definitions included in the definitions of those groups, are as defined herein.

The group attached to the C-terminal analogous portion of the molecule, i.e.,

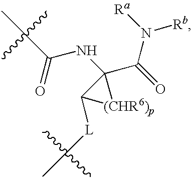

wherein the carboxamide or analog thereof defined by the C(O)NR^{a}R^{b} group attached to the cycloalkyl ring, includes various embodiments. For example, R^{a} and R^{b} can each be hydrogen, in which case the carboxamide is a simple C(O)NH₂ group.

In various other embodiments, one of R^{a} and R^{b} is hydrogen and the other is a carbon-linked group, for example, an aralkyl group such as a phenethyl group, providing an N-phenethylcarboxamide, C(O)NHCH₂CH₂— (phenyl), wherein the phenyl ring can be unsubstituted, or substituted with J groups. More specifically, the phenethyl group can be a 4-methylphenethyl group, a 3,4-dimethylphenethyl group, a 3-chlorophenethyl group, a 4-chlorophenethyl group, a 3-fluorophenethyl group, a 4-fluorophenethyl group, a 2,4-dichlorphenethyl group, a 2,6-dichlorophenethyl group, a 2,4-difluorophenethyl group, or a 2,6-difluorophenethyl group. Or, the carbon linked group can be a heteroarylalkyl group, such as a 4-pyridylethyl group. In various embodiments, mono- and di-substituted carboxamide are provided as are described herein.

In various other embodiments, one of R^{a} and R^{b} can be hydrogen, and the other can be an oxygen-linked group, such as an N-benzyloxy group. It is understood that a group of this general type, C(O)NHO(alkyl) is an O-alkylhydroxamate, so an N-benzyloxycarboxamide is equivalent to an N-benzylhydroxamate. Other embodiments include the hydroxamic acid, (C(O)NHOH, as well as in various embodiments O-cycloalkyl, O-heterocyclyl, O-aryl, O-heteroaryl, and O-acyl hydroxamates.

In various other embodiments, one of R^{a} and R^{b} can be hydrogen, and the other can be a nitrogen-linked group, such as a dialkylamino group. It is understood that a group of this general type, C(O)NHN(alkyl)₂ is an acylhydrazide, and thus various acylhydrazide groups are included in embodiments of the inventive compounds.

In various other embodiments, R^{a} and R^{b} together with the N to which they are bonded form a ring that can include other heteroatoms, can be substituted with substituents as described herein, or can be fused to another ring. For example, R^{a} and R^{b} together with the N to which they are bonded can form a hexahydroazepine, such that the C(O)NR^{a}R^{b} group is an N-acyl amide thereof.

The carboxamide is bonded to a carbon atom that is contained within a cycloalkane ring, the cycloalkane ring itself making up part of the macrocyclic ring that further include a —(CH₂)ₘ-M-CH₂(CH₂)ₙ—CH₂-L-group, bond via the L group to the cycloalkane ring, and bonded at the other end to the D atom, thereby forming the macrocycle. The cycloalkane ring can bear an independently selected R⁶ group on the carbon atom not bonded directly to the L group or to the C(O)NR^{a}R^{b} carboxamide group. The cycloalkane ring has p+2 ring members, including 3-, 4-, 5-, and 6-membered ring sizes in various embodiments. R⁶ can be, for example, hydrogen at every occurrence, thus providing in various embodiments cyclopropyl, cyclobutyl, cyclopentyl and cyclohexyl rings bearing the L group, the carboxamide C(O)NR^{a}R^{b} group, and the nitrogen atom forming an amide with the carboxyl group of the proline-analogous pyrrolidine ring. In other embodiments, R⁶ can be at one occurrence an alkyl group and at all other occurrences hydrogen, providing, for example, a methylcyclopropyl group when p=1.

An embodiment of the invention provides a compound of Formula I wherein D is CH₂ and W-K-V-T is absent. In various embodiments compounds of the invention lack the W-V-K-T “N-terminal” tail and the macrocyclic ring is unsubstituted at that position.

In another embodiment, D is N and V-K are a bond such that T is bonded directly to D. T can be R¹¹, alkyl-R¹¹, alkenyl-R¹¹, alkynyl-R¹¹, OR¹¹, N(R¹¹)₂, C(O)R¹¹, or C(═NOalkyl)R¹¹; wherein R¹¹ is independently at each occurrence hydrogen, alkyl, aryl, aralkyl, alkoxy, amino, alkylamino, dialkylamino, cycloalkyl, cycloalkenyl, [cycloalkyl or cycloalkenyl]-[alkyl or alkenyl], heterocyclyl, heterocyclylalkyl, heterocyclylalkenyl, heteroaryl, heteroarylalkyl, or heteroarylalkenyl, wherein any R¹¹ except hydrogen is substituted with 0-3 J groups, or a first R¹¹ and a second R¹¹ together with a nitrogen atom to which they are bound form a mono- or bicyclic ring system. In various embodiments, T is C(O)R11, providing amide, carbamate (when R¹¹ is alkoxy) and urea (when R¹¹ is amino, alkylamino or dialkylamino) derivatives of the macrocyclic ring including the nitrogen atom.

In another embodiment, D is CH, and W-V-K-T are as defined herein. W can be C(R⁶)₂, O, or NR⁷ when D is CH. In various embodiments, W is C(R⁶)₂, for example W is CH₂. The definitions of X, Y, and Z are the same in embodiments wherein W is C(R⁶)₂ as in embodiments wherein W is a bond (when D is N) or absent (when D is CH₂). For example, in various embodiments, X can be a bond, O, S, CH(R⁶) or N(R⁷), Y is a bond, CH(R⁶), C(O), C(O)C(O), S(O), S(O)₂, or S(O)(NR⁷), provided that when both X and Y are bonds, they together form a single bond, and Z can be hydrogen, alkyl, aryl, aralkyl, cycloalkyl, cycloalkylalkyl, heterocyclyl, heterocyclylalkyl, heteroaryl, heteroarylalkyl, OR⁹, or N(R⁹)₂, wherein any carbon atom is unsubstituted or is substituted with J, and wherein R⁹ is independently at each occurrence hydrogen, alkyl, alkenyl, aryl, aralkyl, aralkenyl, [cycloalkyl or cycloalkenyl]-[alkyl or alkenyl], heterocyclyl, heterocyclylalkyl, heterocyclylalkenyl, heteroaryl, or heteroarylalkyl, or two R⁹ groups together with a nitrogen atom to which they are bound can form together with the nitrogen atom a 5-11 membered mono- or bicyclic heterocyclic ring system substituted with 0-3 J groups and further including 0-3 additional heteroatoms selected from the group consisting of O, NR⁷, S, S(O), and S(O)₂. In other embodiments, Z can be a substituted aryl or heteroaryl group; wherein any aryl or heteroaryl is substituted with 1-3 J groups. In yet other embodiments, Z can be a group of the formula:

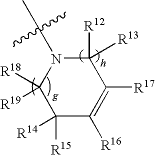

wherein R¹², R¹³, R¹⁴, R¹⁵, R¹⁸ and R¹⁹ can be independently hydrogen, fluorine, or a substituted or unsubstituted alkyl, cycloalkyl, cycloalkenyl, [cycloalkyl or cycloalkenyl]-[alkyl or alkenyl], aryl, aralkyl, aralkenyl, heterocyclyl, heterocyclylalkyl, heterocyclylalkenyl, heteroaryl, heteroarylalkyl, or heteroarylalkenyl group; or R¹² and R¹³ or R¹⁴ and R¹⁵ or R¹⁸ and R¹⁹, together with a carbon atom to which they are attached, can form a C₃₋₆ cycloalkyl group, and R¹⁶ and R¹⁷ can be independently hydrogen, fluorine, or a substituted or unsubstituted alkyl, cycloalkyl, cycloalkenyl, [cycloalkyl or cycloalkenyl]-[alkyl or alkenyl], aryl, aralkyl, aralkenyl, heterocyclyl, heterocyclylalkyl, heterocyclylalkenyl, heteroaryl, heteroarylalkyl, or heteroarylalkenyl group; or R¹⁶ and R¹⁷ together with the atoms to which they are attached form a fused substituted or unsubstituted aryl or heteroaryl group, g is 0-1 and h is 0-2.

In various other embodiments, Z is a group of the formula:

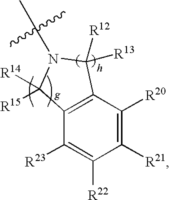

wherein g is 0-2 and h is 0-2, and R¹², R¹³, R¹⁴, and R¹⁵ can be independently at each occurrence hydrogen, fluorine, or a substituted or unsubstituted alkyl, cycloalkyl, cycloalkenyl, [cycloalkyl or cycloalkenyl]-[alkyl or alkenyl], aryl, aralkyl, aralkenyl, heterocyclyl, heterocyclylalkyl, heterocyclylalkenyl, heteroaryl, heteroarylalkyl, or heteroarylalkenyl group; or R¹² and R¹³ or R¹⁴ and R¹⁵, together with a carbon atom to which they are attached, can form a C₃₋₆ cycloalkyl group and R²⁰, R²¹, R²², R²³ are as defined above.

More specifically, Z can be an unsubstituted isoindoline group, or can be an otherwise unsubstituted isoindolidine group bearing a fluorine atom on the phenyl ring, such as in the R²⁰ position.

In various other embodiments, Z is an analog of the isoindolidine group immediately above wherein one of the phenyl ring carbon atoms, such as the ring carbon atom bearing R²⁰, or the ring carbon atom bearing R²¹, is replaced by a nitrogen atom lacking a substituent.

When W is NR⁷, O, or S, other definitions of X, Y, and Z are applicable. For example, X can be O, CH₂, or NR⁷, Y can be C(R⁶)₂ or absent; and Z can be a substituted alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, alkoxy, aryloxy, alkylthio, arylthio, alkylamino, arylamino, heteroaryl, or heteroarylalkyl; wherein any alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, alkoxy, aryloxy, alkylthio, arylthio, alkylamino, arylamino, heteroaryl, or heteroarylalkyl is substituted with 1-3 J groups, provided that K and V are both bonds, taken together forming a single bond such that T is bonded directly to W, T is not C(O)R¹¹.

In various other embodiments, when W is NR7, O, or S, X can be O, Y can be C(O), and Z can be a group of the formula

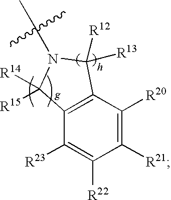

wherein g is 0-2 and h is 0-2, R¹², R¹³, R¹⁴, and R¹⁵ can be independently at each occurrence hydrogen, fluorine, or a substituted or unsubstituted alkyl, cycloalkyl, cycloalkenyl, [cycloalkyl or cycloalkenyl]-[alkyl or alkenyl], aryl, aralkyl, aralkenyl, heterocyclyl, heterocyclylalkyl, heterocyclylalkenyl, heteroaryl, heteroarylalkyl, or heteroarylalkenyl group; or R¹² and R¹³ or R¹⁴ and R¹⁵, together with a carbon atom to which they are attached, can form a C₃₋₆ cycloalkyl group, and R²⁰, R²¹, R²², R²³ can be as defined above.

More specifically, Z can be an unsubstituted isoindoline group, or can be an otherwise unsubstituted isoindolidine group bearing a fluorine atom on the phenyl ring, such as in the R²⁰ position, e.g:

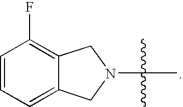

Various embodiments further provide compounds of Formula X, wherein L is C₂H₂, which can be either Z or E substituted (i.e., cis or trans). A compound of Formula X wherein L is C₂H₂ can be prepared by an olefin metathesis cyclization approach, as described below. The olefinic group can be hydrogenated to provide a compound of Formula X wherein L is C₂H₄, or can be dehydrogenated to provide a compound of Formula X wherein L is C₂, using methods well known in the art.

In other embodiments, L can be O or S. Such compounds can be prepared by methods well known in the art, for example by formation of an O or S anion and subsequent displacement of a leaving group on the chain to which the O or S is being coupled.

Various embodiments of the invention also provide compounds of Formula X wherein p is 1, i.e., the ring containing the (CH₂)ₚ moiety is a cyclopropane ring. In particular, when p is 1, L and the ring together can form a vinylcyclopropane moiety. For example, when L and the ring form a vinylcyclopropane moiety, M can be CH₂, m=1, and n=1, whereby a 5-carbon linker chain forms the macrocyclic ring connecting the end of the vinyl group distal from the cyclopropane to the α-carbon of the aminoacid N-terminal to the proline analog ring.

While the inventive compounds include all the stereoisomers of formula X, in a preferred embodiment, the pyrrolidine ring making up the proline analog is substituted with the proline carboxyl group and the 4-substituent (X-Y-Z) being disposed in a trans orientation on the proline ring, thus, a compound of formula XI:

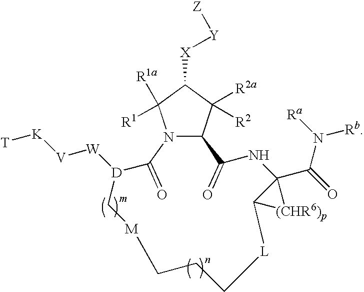

### Methods/Uses

In one aspect, the invention provides methods of inhibiting HCV NS3 protease. The methods include contacting the hepatitis C viral serine protease with a compound as described herein. In other embodiments, the methods of inhibiting HCV NS3 protease include administering a compound as described herein to a subject infected with hepatitis C virus.

In another aspect, the invention provides methods for treating hepatitis C viral infection. The methods include administering to a subject in need of such treatment an effective amount of a compound of the invention as described herein. As used herein, “a compound” can refer to a single compound or a plurality of compounds. In some embodiments, the methods for treating hepatitis C viral infection include administering to a subject in need of such treatment an effective amount of a composition comprising a compound of the invention and a pharmaceutically acceptable carrier.

In another embodiment, the invention provides methods for treating hepatitis C viral infection comprising administering to a subject in need of such treatment an effective amount of a compound of the invention in combination with another anti-viral agent. The term “anti-viral agent” as used herein denotes a compound which interferes with any stage of the viral life cycle to slow or prevent HCV reproduction. Representative anti-viral agents include, without limitation, NS3 protease inhibitors, INTRON-A, (interferon alfa-2b available from Schering Corporation, Kenilworth, N.J.), PEG-INTRON (peginteferon alfa-2b, available from Schering Corporation, Kenilworth, N.J.), ROFERON-A (recombinant interferon alfa-2a available Hoffmann-La Roche, Nutley, N.J.), PEGASYS (peginterferon alfa-2a available Hoffmann-La Roche, Nutley, N.J.), INFERGEN A (Schering Plough, inteferon-alpha 2B+Ribavirin), WELLFERON (interferon alpha-n1), nucleoside analogues, IRES inhibitors, NS5b inhibitors, E1 inhibitors, E2 inhibitors, IMPDH inhibitors, NS5 polymerase inhibitors and/or NTPase/helicase inhibitors. In certain embodiments, the methods of treating HCV infection include administering to a subject in need of such treatment an effective amount of a compound of the invention in combination with another NS3 protease inhibitor. Examples of other NS3 protease inhibitors which can be administered in combination with compounds of the present invention include, without limitation, VX950 and BILN2061 (Lin C, Lin K, Luong Y, Rao B G, Wei Y Y, Brennan D L, Fulghum J R, Hsiao H M, Ma S, Maxwell J P, Cottrell K M, Perni R B, Gates C A, Kwong A D, “In Vitro Resistance Studies of Hepatitis C Virus Serine Protease Inhibitors VX950 and BILN2061”, J. Biol. Chem., 2004, 279, 17508-514).

Still other antiviral agents that may be used in conjunction with inventive compounds for the treatment of HCV infection include, but are not limited to, ribavirin (1-beta-D-ribofuranosyl-1H-1,2,-4-triazole-3-carboxamide, available from ICN Pharmaceuticals, Inc., Costa Mesa, Calif.; described in the Merck Index, entry 8365, Twelfth Edition); REBETROL® (Schering Corporation, Kenilworth, N.J.), COPEGASUS® (Hoffmann-La Roche, Nutley, N.J.); BEREFOR® (interferon alfa 2 available from Boehringer Ingelheim Pharmaceutical, Inc., Ridgefield, Conn.); SUMIFERON® (a purified blend of natural alpha interferons such as Sumiferon available from Sumitomo, Japan); ALFERON® (a mixture of natural alpha interferons made by Interferon Sciences, and available from Purdue Frederick Co., CT); .alpha.-interferon; natural alpha interferon 2a; natural alpha interferon 2b; pegylated alpha interferon 2a or 2b; consensus alpha interferon (Amgen, Inc., Newbury Park, Calif.); VIRAFERON®; INFERGEN®; REBETRON® (Schering Plough, Inteferon-alpha 2B+Ribavirin); pegylated interferon alpha (Reddy, K. R. et al. “Efficacy and Safety of Pegylated (40-kd) Interferon alpha-2a Compared with Interferon alpha-2a in Noncirrhotic Patients with Chronic Hepatitis C (Hepatology, 33, pp. 433-438 (2001); consensus interferon (Kao, J. H., et al., “Efficacy of Consensus Interferon in the Treatment of Chronic Hepatitis” J. Gastroenterol. Hepatol. 15, pp. 1418-1423 (2000); lymphoblastoid or “natural” interferon; interferon tau (Clayette, P. et al., “IFN-tau, A New Interferon Type I with Antiretroviral activity” Pathol. Biol. (Paris) 47, pp. 553-559 (1999); interleukin 2 (Davis, G. L. et al., “Future Options for the Management of Hepatitis C.” Seminars in Liver Disease, 19, pp. 103-112 (1999); Interleukin 6 (Davis et al. “Future Options for the Management of Hepatitis C.” Seminars in Liver Disease 19, pp. 103-112 (1999); interleukin 12 (Davis, G. L. et al., “Future Options for the Management of Hepatitis C.” Seminars in Liver Disease, 19, pp. 103-112 (1999); and compounds that enhance the development of type 1 helper T cell response (Davis et al., “Future Options for the Management of hepatitis C.” Seminars in Liver Disease, 19, pp. 103-112 (1999)). Also included are compounds that stimulate the synthesis of interferon in cells (Tazulakhova, E. B. et al., “Russian Experience in Screening, analysis, and Clinical Application of Novel Interferon Inducers” J. Interferon Cytokine Res., 21 pp. 65-73) including, but are not limited to, double stranded RNA, alone or in combination with tobramycin, and Imiquimod (3M Pharmaceuticals; Sauder, D. N. “Immunomodulatory and Pharmacologic Properties of Imiquimod” J. Am. Acad. Dermatol., 43 pp. S6-11 (2000)

In another embodiment, the invention provides a method for treating hepatitis C viral infection, comprising administering to a subject in need of such treatment an effective amount of a compound of the invention in combination with an anti-proliferative agent. The term “anti-proliferative agent” as used herein denotes a compound which inhibits cellular proliferation. Cellular proliferation can occur, for example without limitation, during carcinogenesis, metastasis, and immune responses. Representative anti-proliferative agents include, without limitation, 5-fluorouracil, daunomycin, mitomycin, bleomycin, dexamethasone, methotrexate, cytarabine, mercaptopurine.

In another embodiment, the invention provides a method for treating hepatitis C viral infection, comprising administering to a subject in need of such treatment an effective amount of a compound of the invention in combination with an immune modulator. The term “immune modulator” as used herein denotes a compound or composition comprising a plurality of compounds which changes any aspect of the functioning of the immune system. In this context, immune modulator includes without limitation anti-inflammatory agents and immune suppressants. Representative immune modulator include without limitation steroids, non-steroidal anti-inflammatories, COX2 inhibitors, anti-TNF compounds, anti-IL-1 compounds, methotrexate, leflunomide, cyclosporin, FK506 and combinations of any two or more thereof. Representative steroids in this context include without limitation prednisone, prednisolone, and dexamethasone. Representative non-steroidal anti-inflammatory agents in this context include without limitation ibuprofen, naproxen, diclofenac, and indomethacin. Representative COX2 inhibitors in this context include without limitation rofecoxib and celecoxib. Representative Anti-TNF compounds in this context include without limitation enbrel, infliximab, and adalumimab. Representative anti-IL-1 compounds in this context include without limitation anakinra. Representative immune suppressants include without limitation cyclosporin and FK506.

Compounds of the invention include mixtures of stereoisomers such as mixtures of diastereomers and/or enantiomers. In some embodiments, the compound, e.g. of Formula X, is 90 weight percent (wt %) or greater of a single diastereomer of enantiomer. In other embodiments, the compound is 92, 94, 96, 98 or even 99 wt % or more of a single diastereomer or single enantiomer.

A variety of uses of the invention compounds are possible along the lines of the various methods of treating a subject as described above. Exemplary uses of the invention methods include, without limitation, use of a compound of the invention in a medicament or for the manufacture of a medicament for treating a condition that is regulated or normalized via inhibition of the HCV NS3 serine protease.

### Biochemical Methods

Fluorescence resonance energy transfer (FRET; see e.g., Heim et al., (1996) Curr. Biol. 6:178-182; Mitra et al., (1996) Gene 173:13-17; and Selvin et al., (1995) Meth. Enzymol. 246:300-345) is an exquisitely sensitive method for detecting energy transfer between two fluorophoric probes. As known in the art, such probes are given the designations “donor” and “acceptor” depending on the relative positions of the maxima in the absorption and emission spectra characterizing the probes. If the emission spectrum of the acceptor overlaps the absorption spectrum of the donor, energy transfer can occur. Because of the known and highly non-linear relationship of energy transfer and distance between fluorophores, approximated by an inverse sixth power dependence on distance, FRET measurements correlate with distance. For example, when the probes are in proximity, such as when the probes are attached to the N- and C-termini of a peptide substrate, and the sample is illuminated in a spectrofluorometer, resonance energy can be transferred from one excited probe to the other resulting in observable signal. Upon scission of the peptide linking the probes, the average distance between probes increases such that energy transfer between donor and accept probe is not observed. As a result, the degree of hydrolysis of the peptide substrate, and the level of activity of the protease catalyzing hydrolysis of the peptide substrate, can be quantitated. Accordingly, using methods known in the arts of chemical and biochemical kinetics and equilibria, the effect of inhibitor on protease activity can be quantitated.

### Compositions and Combination Treatments

### A. Compositions.

Another aspect of the invention provides compositions of the compounds of the invention, alone or in combination with another NS3 protease inhibitor or another type of antiviral agent and/or another type of therapeutic agent. As set forth herein, compounds of the invention include stereoisomers, tautomers, solvates, prodrugs, pharmaceutically acceptable salts and mixtures thereof. Compositions containing a compound of the invention may be prepared by conventional techniques, e.g. as described in Remington: The Science and Practice of Pharmacy, 19th Ed., 1995. The compositions may appear in conventional forms, for example capsules, tablets, aerosols, solutions, suspensions or topical applications.

Typical compositions include a compound of the invention which inhibits the enzymatic activity of the HCV NS3 protease, and a pharmaceutically acceptable excipient which may be a carrier or a diluent. For example, the active compound will usually be mixed with a carrier, or diluted by a carrier, or enclosed within a carrier which may be in the form of an ampoule, capsule, sachet, paper, or other container. When the active compound is mixed with a carrier, or when the carrier serves as a diluent, it may be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent may include any sustained release material known in the art, such as glyceryl monostearate or glyceryl distearate, alone or mixed with a wax.

The formulations can be mixed with auxiliary agents which do not deleteriously react with the active compounds. Such additives can include wetting agents, emulsifying and suspending agents, salt for influencing osmotic pressure, buffers and/or coloring substances preserving agents, sweetening agents or flavoring agents. The compositions can also be sterilized if desired.

The route of administration may be any route which effectively transports the active compound of the invention which inhibits the enzymatic activity of the HCV NS3 protease to the appropriate or desired site of action, such as oral, nasal, pulmonary, buccal, subdermal, intradermal, transdermal or parenteral, e.g., rectal, depot, subcutaneous, intravenous, intraurethral, intramuscular, intranasal, ophthalmic solution or an ointment, the oral route being preferred.

If a solid carrier is used for oral administration, the preparation may be tabletted, placed in a hard gelatin capsule in powder or pellet form or it can be in the form of a troche or lozenge. If a liquid carrier is used, the preparation may be in the form of a syrup, emulsion, soft gelatin capsule or sterile injectable liquid such as an aqueous or non-aqueous liquid suspension or solution.

Injectable dosage forms generally include aqueous suspensions or oil suspensions which may be prepared using a suitable dispersant or wetting agent and a suspending agent Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Preferably, the oil or fatty acid is non-volatile, including natural or synthetic oils, fatty acids, mono-, di- or tri-glycerides.

For injection, the formulation may also be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these. The compounds may be formulated for parenteral administration by injection such as by bolus injection or continuous infusion. A unit dosage form for injection may be in ampoules or in multi-dose containers. The formulations of the invention may be designed to provide quick, sustained, or delayed release of the active ingredient after administration to the patient by employing procedures well known in the art. Thus, the formulations may also be formulated for controlled release or for slow release.

Compositions contemplated by the present invention may comprise, for example, micelles or liposomes, or some other encapsulated form, or may be administered in an extended release form to provide a prolonged storage and/or delivery effect. Therefore, the formulations may be compressed into pellets or cylinders and implanted intramuscularly or subcutaneously as depot injections or as implants such as stents. Such implants may employ known inert materials such as silicones and biodegradable polymers, e.g., polylactide-polyglycolide. Examples of other biodegradable polymers include poly(orthoesters) and poly(anhydrides).

For nasal administration, the preparation may contain a compound of the invention which inhibits the enzymatic activity of the HCV NS3 protease, dissolved or suspended in a liquid carrier, preferably an aqueous carrier, for aerosol application. The carrier may contain additives such as solubilizing agents, e.g., propylene glycol, surfactants, absorption enhancers such as lecithin (phosphatidylcholine) or cyclodextrin, or preservatives such as parabens.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet that may be prepared by conventional tabletting techniques may contain:

Core:

| | |
|---|---|
| Active compound (as free compound or salt thereof) | 250 mg |
| Colloidal silicon dioxide (Aerosil) ® | 1.5 mg |
| Cellulose, microcryst. (Avicel) ® | 70 mg |
| Modified cellulose gum (Ac-Di-Sol) ® | 7.5 mg |
| Magnesium stearate | Ad. |

Coating:

| | |
|---|---|
| HPMC approx. | 9 mg |
| *Mywacett 9-40 T approx. | 0.9 mg |

*Acylated monoglyceride used as plasticizer for film coating.

A typical capsule for oral administration contains compounds of the invention (250 mg), lactose (75 mg) and magnesium stearate (15 mg). The mixture is passed through a 60 mesh sieve and packed into a No. 1 gelatin capsule. A typical injectable preparation is produced by aseptically placing 250 mg of compounds of the invention into a vial, aseptically freeze-drying and sealing. For use, the contents of the vial are mixed with 2 mL of sterile physiological saline, to produce an injectable preparation.

The compounds of the invention may be administered to a mammal, especially a human in need of such treatment, prevention, elimination, alleviation or amelioration of the various diseases as mentioned above, e.g., HCV infection. Such mammals include also animals, both domestic animals, e.g. household pets, farm animals, and non-domestic animals such as wildlife.

The compounds of the invention are effective over a wide dosage range. For example, in the treatment of adult humans, dosages from about 0.05 to about 5000 mg, preferably from about 1 to about 2000 mg, and more preferably between about 2 and about 2000 mg per day may be used. A typical dosage is about 10 mg to about 1000 mg per day. In choosing a regimen for patients it may frequently be necessary to begin with a higher dosage and when the condition is under control to reduce the dosage. The exact dosage will depend upon the activity of the compound, mode of administration, on the therapy desired, form in which administered, the subject to be treated and the body weight of the subject to be treated, and the preference and experience of the physician or veterinarian in charge. HCV NS3 protease inhibitor activity of the compounds of the invention may be determined by use of an in vitro assay system which measures the potentiation of inhibition of the HCV NS3 protease. Inhibition constants (i.e., Kᵢ or IC₅₀ values as known in the art) for the HCV NS3 protease inhibitors of the invention may be determined by the method described in the Examples.

Generally, the compounds of the invention are dispensed in unit dosage form comprising from about 0.05 mg to about 1000 mg of active ingredient together with a pharmaceutically acceptable carrier per unit dosage.

Usually, dosage forms suitable for oral, nasal, pulmonal or transdermal administration comprise from about 125 μg to about 1250 mg, preferably from about 250 μg to about 500 mg, and more preferably from about 2.5 mg to about 250 mg, of the compounds admixed with a pharmaceutically acceptable carrier or diluent.

The invention also encompasses prodrugs of a compound of the invention which on administration undergo chemical conversion by metabolic or other physiological processes before becoming active pharmacological substances. Conversion by metabolic or other physiological processes includes without limitation enzymatic (e.g, specific enzymatically catalyzed) and non-enzymatic (e.g., general or specific acid or base induced) chemical transformation of the prodrug into the active pharmacological substance. In general, such prodrugs will be functional derivatives of a compound of the invention which are readily convertible in vivo into a compound of the invention. Conventional procedures for the selection and preparation of suitable prodrug derivatives are described, for example, in Design of Prodrugs, ed. H. Bundgaard, Elsevier, 1985.

In another aspect, there are provided methods of making a composition of a compound described herein comprising formulating a compound of the invention with a pharmaceutically acceptable carrier or diluent. In some embodiments, the pharmaceutically acceptable carrier or diluent is suitable for oral administration. In some such embodiments, the methods may further comprise the step of formulating the composition into a tablet or capsule. In other embodiments, the pharmaceutically acceptable carrier or diluent is suitable for parenteral administration. In some such embodiments, the methods further comprise the step of lyophilizing the composition to form a lyophilized preparation.

### B. Combinations.

The compounds of the invention may be used in combination with i) one or more other NS3 protease inhibitors and/or ii) one or more other types of antiviral agents (employed to treat viral infection and related diseases) and/or one or more other types of therapeutic agents which may be administered orally in the same dosage form, in a separate oral dosage form (e.g., sequentially or non-sequentially) or by injection together or separately (e.g., sequentially or non-sequentially).

Accordingly, in another aspect the invention provides combinations, comprising:

a) a compound of the invention as described herein; and
b) one or more compounds comprising:

i) other compounds of the present invention

ii) anti-viral agents including, but not limited to, other NS3 protease inhibitors

iii) anti-proliferative agents

iv) immune modulators.

Combinations of the invention include mixtures of compounds from (a) and (b) in a single formulation and compounds from (a) and (b) as separate formulations. Some combinations of the invention may be packaged as separate formulations in a kit. In some embodiments, two or more compounds from (b) are formulated together while a compound of the invention is formulated separately.

Combinations of the invention can further comprise a pharmaceutically acceptable carrier. In some embodiments, the compound of the invention is 90 wt % or more of a single diastereomer or single enantiomer. Alternatively, the compound of the invention can be 91, 92, 93, 94, 95, 96, 97, 98, or 99 wt % or more of a single diastereomer or single enantiomer.

The dosages and formulations for the other antiviral agent to be employed, where applicable, will be as set out in the latest edition of the Physicians' Desk Reference.

In carrying out the methods of the invention, a composition may be employed containing the compounds of the invention, with or without another antiviral agent and/or other type therapeutic agent, in association with a pharmaceutical vehicle or diluent. The composition can be formulated employing conventional solid or liquid vehicles or diluents and pharmaceutical additives of a type appropriate to the mode of desired administration. The compounds can be administered to mammalian species including humans, monkeys, dogs, etc. by an oral route, for example, in the form of tablets, capsules, granules or powders, or they can be administered by a parenteral route in the form of injectable preparations. The dose for adult humans is preferably between 10 and 1,000 mg per day, which can be administered in a single dose or in the form of individual doses from 1-4 times per day.

### Methods of Synthesis

Embodiments of compounds of formula X of the invention can be prepared according to embodiments of synthetic methods of the invention. For example, compound Y can be prepared by an olefin metathesis reaction using a transition metal catalyst, as shown in the following scheme:

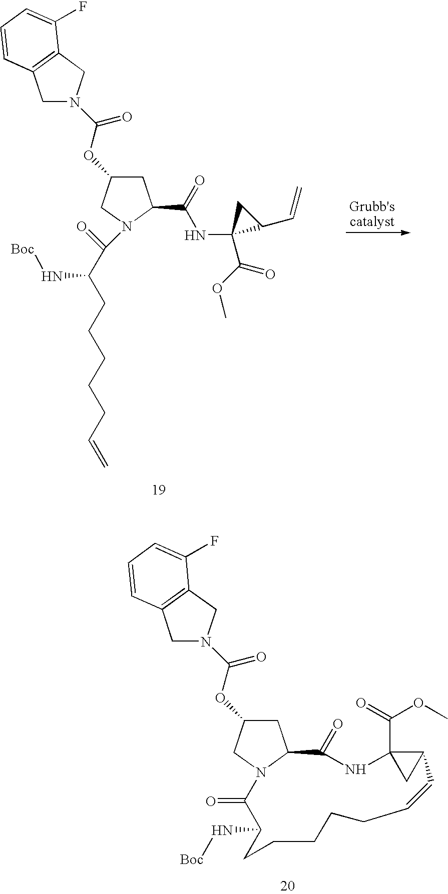

using Grubb's catalyst or the like in an inert solvent such as dichloromethane. The solution can be deoxygenated before carrying out the metathesis reaction.

Accordingly, a general method of synthesis of embodiments of compounds of the invention provides a method of preparing a compound of formula X, comprising contacting a compound of formula XII:

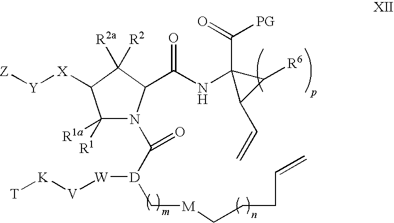

with a transition metal catalyst in an amount, at a temperature, and for a duration effective to form the compound of formula XIII

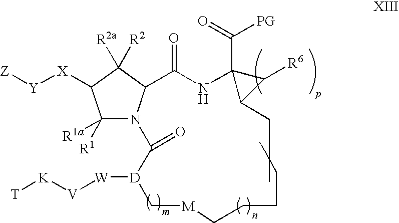

wherein PG is a carboxyl protecting group, then, converting PG to NR^{a}R^{b} to provide a compound of formula X of claim 1 wherein L is C₂H₂.

For example, the transition metal catalyst can be Grubb's catalyst, benzylidene-bis(tricyclohexylphosphine)dichlororuthenium.

### EXAMPLES

The following abbreviations are used throughout this document.

| | |
|---|---|
| BOP | Benzotriazol-1-yl-oxy-tris- |
| | (dimethylamino)phosphonium |
| | hexa-fluorophosphate |
| CDI | Carbonyl diimidazole |
| DBU | Diazabicycloundecane |
| DCM | Dichloromethane |
| DIEA, ⁱPr₂EtN | N,N-Diisoproylethylamine |
| DMAP | 4-(N,N-dimethylamino)pyridine |
| DMF | N,N-Dimethylformamide |
| DMSO | Dimethylsulfoxide |
| EDC | 1-Ethyl-3-[3-dimethylaminopropyl]carbodiimide |
| | hydrochloride |
| EtOAc | Ethyl acetate |
| HATU | O-(7-Azabenzotriazole-1-yl)-N,N,N′N′- |
| | tetramethyluronium hexafluorophosphate |
| HOAT | Hydroxyazabenztriazole |
| HOBT | Hydroxybenzotriazole |
| MS | Mass spectroscopy |
| MeOH | Methanol |
| NaH | Sodium hydride |
| NMM | N-Methylmorpholine |
| THF | Tetrahydrofuran |

The compounds and processes of the present invention will be better understood in connection with the following examples, which are intended as an illustration only and not to limit the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Compounds of formula X, wherein common terms are as defined above, may be conveniently prepared by the process outlined in Scheme 1 below.

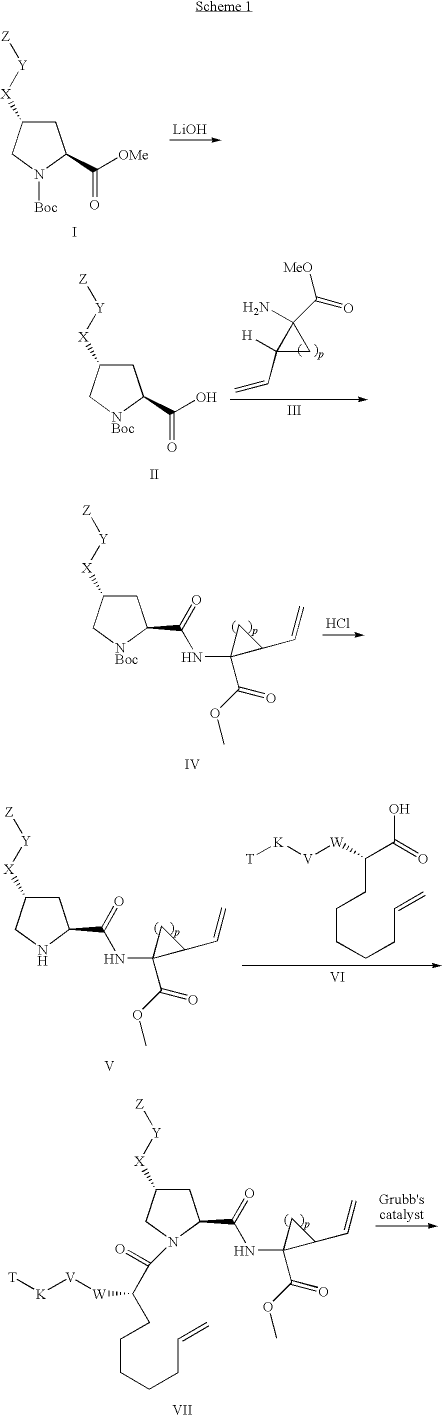

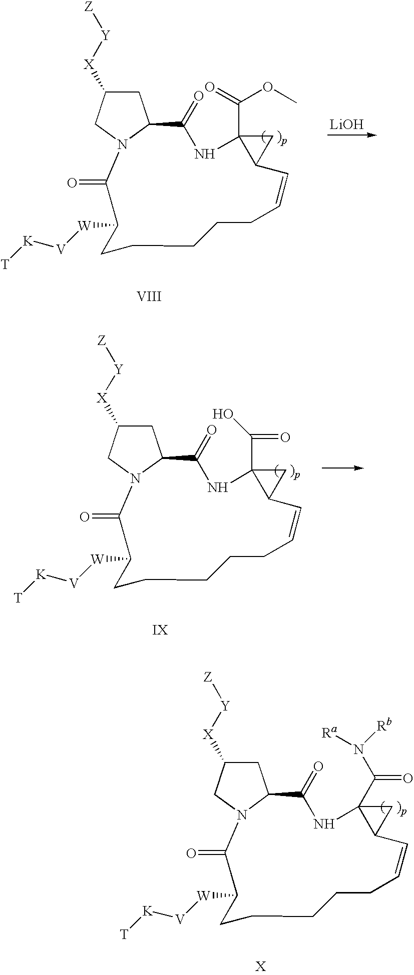

Generally, the processes for preparing the compounds of formula X where X, Y, Z, W, V, K, and T are as defined above comprise the steps of:

a) hydrolyzing a compound of formula I with lithium hydroxide;
b) coupling a compound of formula II with an amino acid of formula III;
c) removing a nitrogen protecting group from a compound of formula IV;
d) coupling a compound of formula V with an amino acid of formula VI to produce a compound of formula VII;
e) forming a macrocycle by reaction a compound of formula VII with a metathesis catalyst such as Grubb's catalyst;
f) hydrolyzing a compound of formula VIII with lithium hydroxide to provide a compound of formula IX; and,
g) forming an amide of the compound of formula IX to provide the inventive compound of formula X.

A particular example of a compound 6 of formula VI was prepared as outlined in Scheme 2.

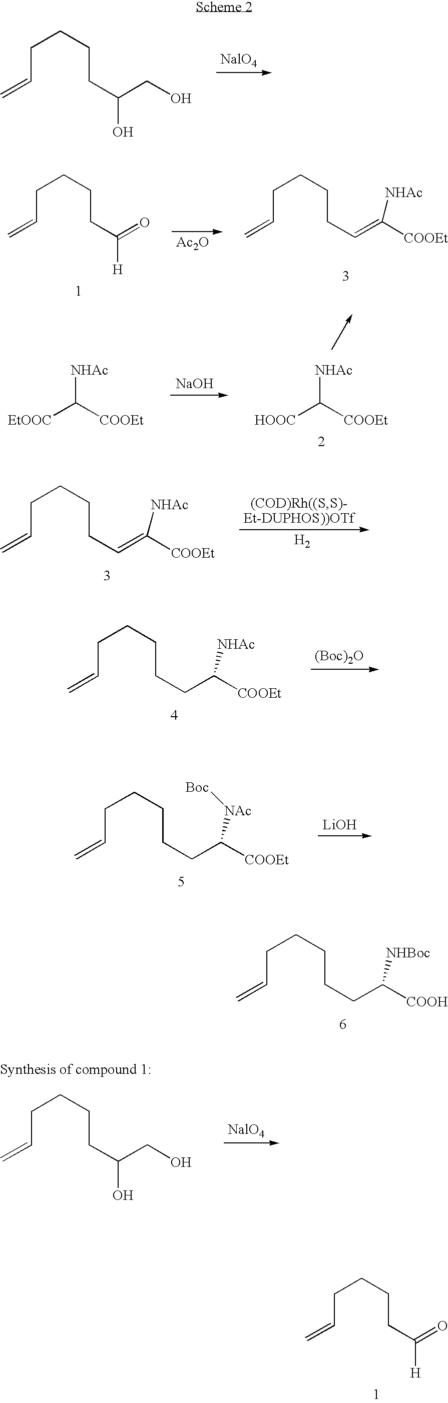

To a magnetically stirred emulsion of commercially available 7-octene-1,2-diol (5 g, 34.7 mmol) and H₂O (20 mL), an aqueous solution of NaIO₄ (8.14 g, 38.2 mmol, in 47.5 mL H₂O) was added over a period of 45 min (slight exotherm observed). The resulting mixture was stirred at room temperature for an additional 1.5 hour (completion of reaction confirmed by TLC). The mixture was then decanted in a separatory funnel and the layers were separated. The organic fraction was dried with sodium sulfate and filtered over a cotton plug (in a Pasteur pipette) to give compound 1 (2.99 g). The aqueous solution was saturated with NaCl, extracted with DCM, dried with anhydrous MgSO4, and concentrated under reduced pressure (without heating, heptenal b.p. 153° C.) to obtain an additional amount of compound 1 (0.855 g). The two fractions were combined to afford the title compound 1 (3.85 g) as a colorless oil.

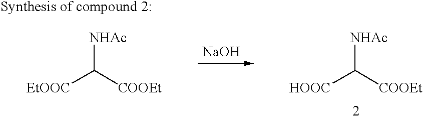

To a stirred solution of diethyl 2-acetamidomalonate (10 g, 46 mmol) in dioxane (60 mL) was added aqueous sodium hydroxide (1 M, 46.5 mL) dropwise over 2 h. The resulting mixture was stirred at room temperature for 15 h, then dioxane was evaporated under reduced pressure, the aqueous solution was washed with three portions of 30 mL of EtOAc and filtered. The filtrate was cooled down to 0° C. and acidified to pH=1 with concentrated HCl (5 mL). After the appearance of a few crystals, the mixture was sonicated and an abundant precipitate appeared. Filtration and drying under reduced pressure afforded the titled compound 2 (7.084 g) as a white solid.

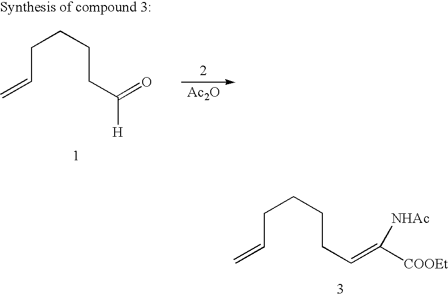

To solid ethyl 2-acetamidomalonate 2 (3.78 g, 20 mmol) was added 1 (2.24 g, 20 mmol.) in solution in pyridine (16 mL). The resulting solution was cooled in a −15° C. bath (KCl/ice) and acetic anhydride (6 mL) was added over 12 min. The resulting orange solution was stirred for 3 h at room temperature and another portion of ethyl 2-acetamidomalonate 2 (1.14 g) was added. The resulting mixture was stirred at room temperature for an extra 15 h. Ice (25 g) was then added and the solution was stirred for 1.5 h, then the mixture was diluted with 100 mL of water and extracted with two portions (75 mL) of ether. The etheral solution was washed with 1N HCl (30 mL), sat. NaHCO₃ (30 mL) and brine (30 mL), dried with Na₂SO₄, concentrated to afford an orange oil (3.71 g) and purified by flash chromatography (EtOAc: hexane=2:3) to give 3 (2.33 g) as a pale yellow oil.

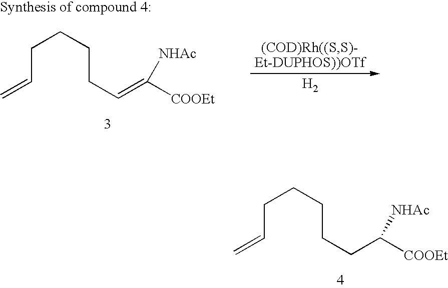

To a degassed (argon bubbling for 30 min.) solution of Z-ethyl 2-acetamido-2,8-nonadienoate 3 (2.73 g, 11.34 mmol) in dry ethanol (20 mL) was added (S,S)-Me-DUPHOS Rh(COD)OTf (9.6 mg, S/C=857). The mixture was put under 45 psi of hydrogen (after 4 vacuum-H₂ cycles) and stirred for 18 h. The resulting mixture was concentrated under reduced pressure to afford the desired compound 4 (2.74 g), which was used in the subsequent step without purification.

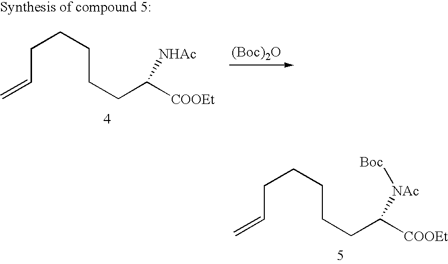

To a solution of crude (S)-ethyl 2-acetamido-8-nonenoate 4 (1.38 g, 5.71 mmol) in THF (16 mL) were added Boc₂O (2.49 g, 2 eq.) and DMAP (139.5 mg, 0.2 eq.), the resultant reaction mixture was heated to reflux for 3.5 h. The reaction mixture was concentrated, diluted with DCM (50 mL), washed with HCl (1 N) (20 mL), brine (15 mL), then saturated aqueous NaHCO₃ (20 mL), dried with MgSO₄. The resulting solution was concentrated under reduced pressure. This compound was used to the next step without further purification.

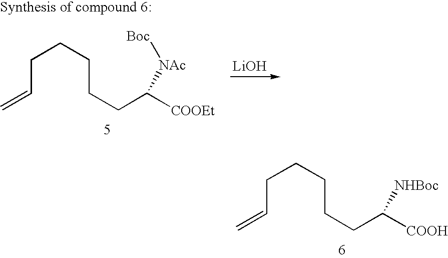

The crude product of 5 was then diluted with THF (12 mL) and water (7.5 mL), LiOH.H₂O (0.48 g, 2 eq.) was added and the resulting mixture was stirred at rt for 18 h (completion of the hydrolysis was confirmed by TLC). The reaction mixture was concentrated under reduced pressure, then diluted with DCM (50 mL), washed with HCl (1 N) (15 mL), dried with anhydrous Na₂SO₄ and concentrated under reduced pressure. This crude product was purified by flash column chromatography (EtOAc:hexane=0:100 to 100:0). The titled compound 6 was obtained as a pale yellow oil (697 mg). LC-MS (ESI, positive): 272 [M+H]⁺.

A particular example of a compound 11 of formula III was prepared as outlined in Scheme 3.

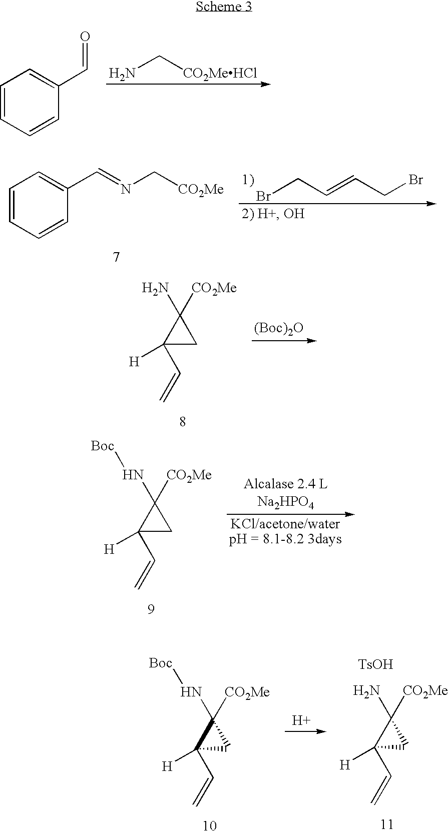

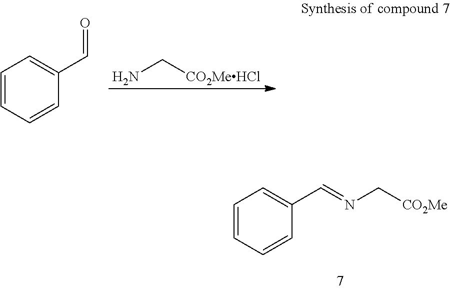

Glycine methyl ester hydrochloride (37.8 g, 300 mmol) was suspended in CH₂Cl₂ (300 mL) in a 1 L of flask. The benzaldehyde (31.8 g, 330 mmol) was added to the reaction mixture. After adding of anhydrous MgSO₄ (21.6 g, 180 mmol), the resultant suspension was cooled in ice to an internal temperature lower than 5° C., and triethylamine (45.6 g, 450 mmol) was added dropwise over 10 min with vigorous stirring. The mixture was then stirred 24 h at room temperature. The mixture was filtered, and the filtrate was evaporated under reduced pressure. The residue was dried to a constant weight under high vacuum to give the desired crude imine as an yellow oil that was used directly in the next step. 52 g of the compound 7 was obtained.

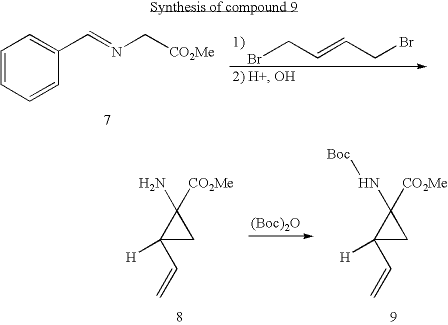

t-BuOLi (45.7 g, 571 mmol) was suspended in toluene (400 mL) at room temperature. A freshly prepared mixture of 7 (50 g, 286 mmol) and 1,4-dibromo-butene (57 g, 272 mmol) in toluene (200 mL) was added dropwise over 30 min to the stirred suspension of the base. After stirring for 60 min at rt, the reaction was quenched by addition of water (100 mL), the organic phase was extracted with TBME (500 mL). The organic phase was mixed with 1N HCl (200 mL) and stirred for 2 h at room temperature to hydrolysis of the intermediate imine. The organic phase was separated and extracted with water (2*200 mL). The combined aqueous phase were mixed with NaCl (250 g) and TBME (300 mL), and 10N NaOH (30 mL) was added dropwise to bring the pH to 12-13. The organic phase was separated and the aqueous phase extracted with additional TBME (3*200 mL). The combined organic extracts containing compound 8 were mixed with Boc₂O (25 g, 115 mmol), and the solution stirred overnight at room temperature. The mixture was then heated to 60° C. for 2 h. The cooled solution was then dried over Na₂SO₄, and concentrated under reduced pressure. The residue was purified by flash column (P:E=25:1 to 15:1) to give 33 g of compound 9. LC-MS (ESI, positive): 242 [M+H]⁺.

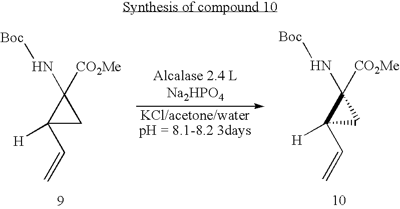

A reactor was charged with Na₂HPO₄ (7.6 g, 55 mmol), water (220 mL) and Alcalase 2.4 L (11 mL). The pH was adjusted to 8.15 by additional Na₂HPO₄ (188 mg, 1.3 mmol). Racemic 9 (7.3 g, 30 mmol) in acetone (15 mL) was added and the mixture was stirred at 40° C. while maintaining the pH at 8.15 by periodic addition of 1N NaOH (20 mL). Enantiomeric purity of the remaining ester was monitored by HPLC analysis. Heating was discontinued after 70 h and TBME (3*100 mL) was added to extract the resolved ester. The extract was washed with water (2*50 mL), concentrated under vacuum and used directly in the next step. 3.77 g of compound 10 was produced. LC-MS (ESI, positive): 242 [M+H]⁺.

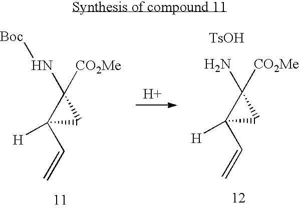

The 11 (3.77 g, 16 mmol) was added into a reactor and most of the solvent removed in vacuum. MIBK (4 mL) was added and warmed to 40° C. P-TsOH (4.46 g, 23 mmol) in a mixture of MeOH (0.9 mL) and MIBK (4 mL) was added and the mixture stirred for 2 h. The mixture was then cooled to 3-8° C. and stirred for an additional 2 h. The product was isolated by filtration, washed with MIBK (30 mL) to give 4.1 g of compound 12. LC-MS (ESI, positive): 142 [M+H]⁺.

A particular example of a compound 21 of formula IX was prepared as outlined in Scheme 4.

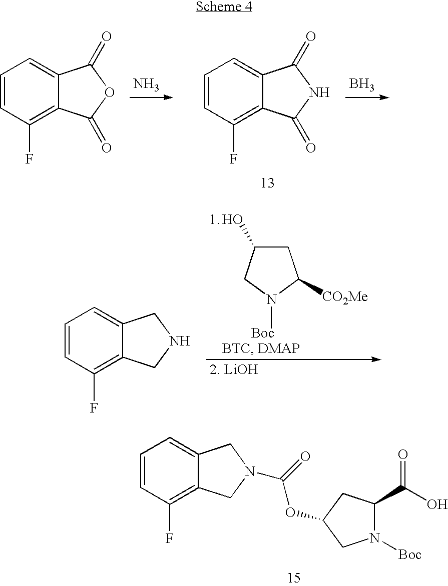

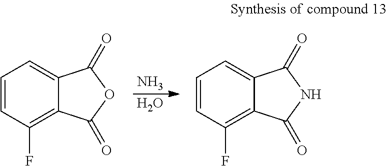

In a 50 ml flask was placed 3-Fluorophthalic anhydride (1.0 g, 6 mmol) and aqueous NH₃ (1.6 g, 24 mmol). The mixture was heated to 280° C. within 30 minutes and then the flask was cooled to room temperature. 0.93 g of compound 13 were isolated as a yellow solid. LC-MS (ESI, positive): 166 [M+H]⁺.

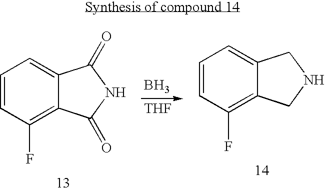

To compound 13 (4.0 g, 24.2 mmol) in a round bottom flask was added 1 M BH₃ in THF solution (97 mL, 97 mmol) dropwise at room temperature. The resulting solution was warmed to reflux for 18 hours. Then the reaction mixture was cooled to 0° C. and methanol (3.1 g, 97 mmol) was added dropwise. The resulting mixture was warmed up to room temperature and then 6 M HCl was added dropwise to adjust the reaction pH to 3, followed by refluxed for 1 hour. After the reaction was completed, the solvents were removed under reduced pressure to give an brown oil. The residue was washed with Et₂O (2×50 ml) and CH₂Cl₂ (2×50 mL). The aqueous phase was adjusted to pH 11 with NaOH. Then the aqueous layer was extracted with ether (4×50 mL), dried over Na₂SO₄, and filtered. Solvents were removed under reduced pressure to give a dark red residue. The pure compound was purified by distillation (2 mmHg, 45° C.) to give compound 14 (1.2 g).

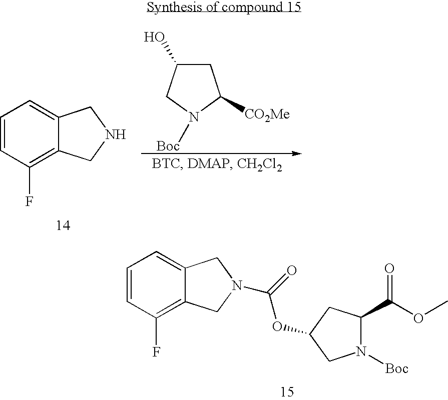

A solution of 14 (4.113 g, 16.8 mmol) and DMAP (3.072 g, 25 mmol) in dichloromethane (17 mL) was added directly to a solution of BTC (1.994 g, 6.7 mmol) in dichloromethane (17 mL) at 0° C. After the addition was finished, the reaction mixture was stirred at room temperature for 3 hrs. Then the mixture was cooled to 0° C. and a solution of DMAP (3.072 g, 25 mmol) in dichloromethane (17 mL) and a solution of Boc trans-hydroxyproline methylester (2.3 g, 16.8 mmol) in dichloromethane (17 mL) were added sequentially. The reaction mixture was stirred at room temperature overnight. Dichloromethane (100 mL) was added to the reaction mixture and the organic layer was washed with 1N HCl (50 mL), saturated aqueous NaHCO₃ (50 mL) and brine (50 mL). It was dried over anhydrous Na₂SO₄ and filtered. After removal of solvents under reduced pressure, the residue was purified by silica gel column chromatography (elution solvent system PE:EA=2:1 to 1:3) to get the compound 15 (3.8 g). LC-MS (ESI, positive): 409 [M+H]⁺.

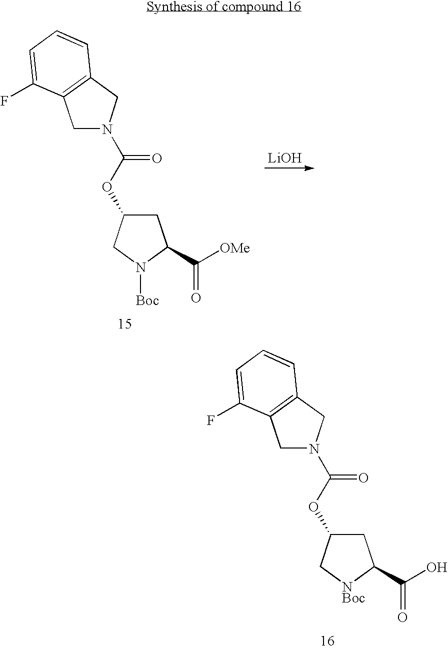

Compound 15 (1.7 g, 4.1 mmol) was dissolved in THF (10 mL), aqueous LiOH (0.5 N, 16 ml) was added and the resulting solution was stirred for 3 h at room temperature. Evaporated most of THF and adjusted the value of pH to 3 with 1N HCl (10 mL) and extracted with DCM (60 mL), combined the organic phase, dried over anhydrous Na₂SO₄, filtrated the Na₂SO₄, and evaporated the solvent, 1.6 g of compound 16 was produced. LC-MS (ESI, positive): 395 [M+H]⁺.

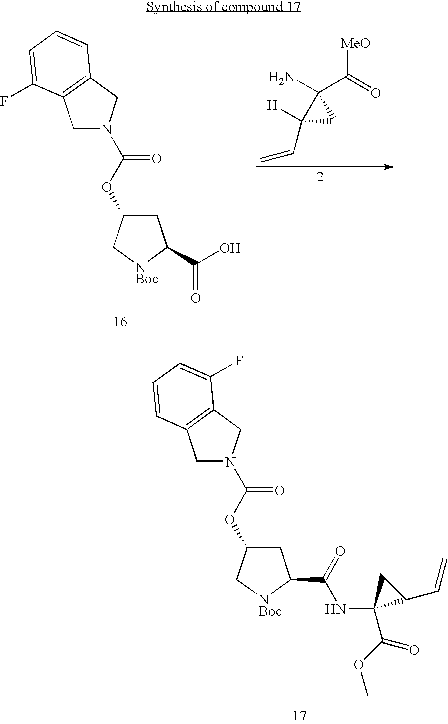

The solution of 16 (1.6 g, 4 mmol) in DCM (10 mL) was added HATU (2.2 g, 6 mmol). 2 (1.9 g, 6 mmol) in DCM (5 mL) was added DIPEA (5 g, 40 mmol), the resulting solution was added into the solution of 16, the reaction mixture was concentrated to dryness, the residue, diluted with EA (50 mL), wash with saturated NaHCO₃ (20 mL) and brine (20 mL) in sequence, dried over anhydrous Na₂SO₄, filtered and concentrated under vacuum. The residue was purified by column chromatography to provide 1.7 g of 17. LC-MS (ESI, positive): 518 [M+H]⁺.

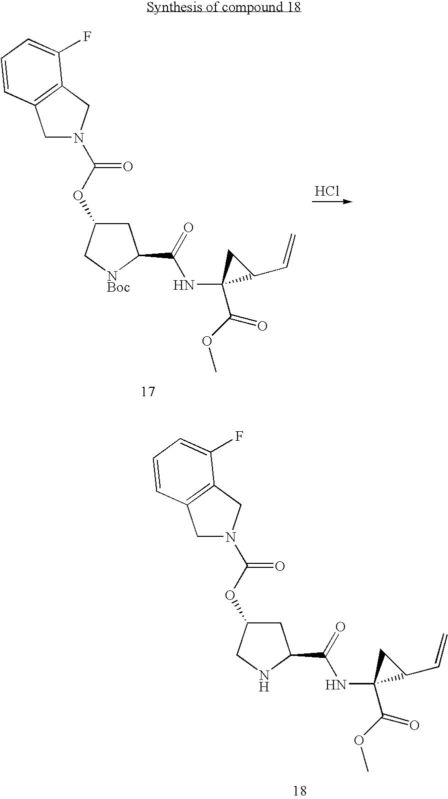

2 ml of TFA was added to a solution of 17 (450 mg, 0.87 mmol) in DCM (5 mL) and the resulting mixture was stirred at rt for 2 h. After the removal of the solvent and TFA under reduced pressure, the residue containing compound 18 was used directly to the next step. LC-MS (ESI, positive): 418 [M+H]⁺.

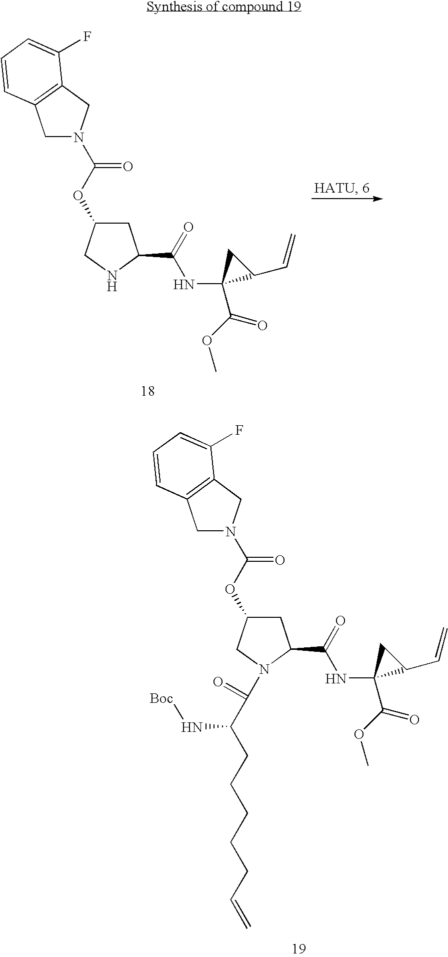

The resulting amine intermediate 18 was then dissolved in a mixture of DCM (5 mL) and DIPEA (561 mg, 5 eq) (solution A). Separately, a mixture of 6 (330 mg, 1.2 mmol), HATU (496 mg, 1.5 eq.) and DIPEA (561 mg, 5 eq) in DCM (5 mL) were allowed to react for 10-20 min. To the resulting mixture was added to solution A dropwise and the resulting solution was left to stir at rt for 3 h. The reaction solution was then concentrated under reduced pressure, diluted with EtOAc (50 mL), washed with aqueous HCl (0.5 N) (20 mL), water (20 mL) and NaHCO₃ (sat.) (20 mL), dried with MgSO₄, and concentrated under reduced pressure. The resulting yellow oil was purified by flash column chromatography (EtOAc:hexane=4:3) to afford 18 (500 mg) as a white foam. LC-MS (ESI, positive): 671 [M+H]⁺.

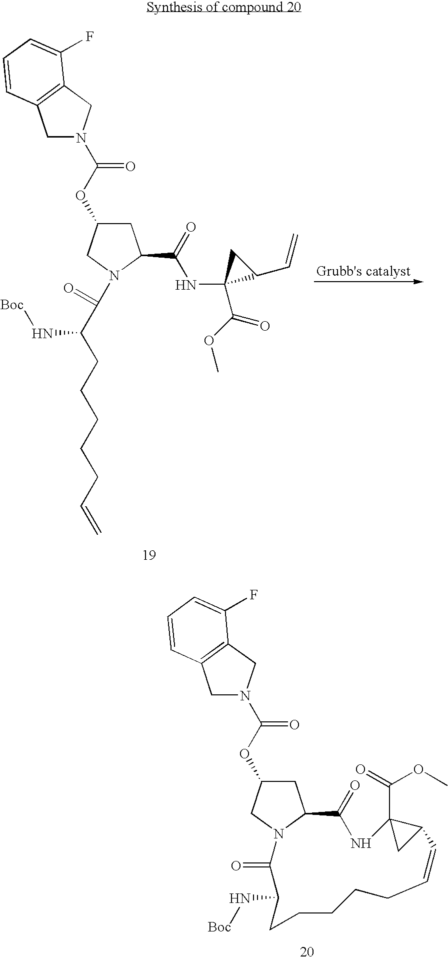

A solution of 19 (500 mg, 0.75 mmol) in dry DCM (20 ml) was deoxygenated (bubbling Ar for 2 h). Grubb's catalyst (22 mg, 5 mol %) was then added as a solid and the reaction was refluxed under argon. After 24 h, the red-orange solution was evaporated to an amorphous residue which was then purified by flash column chromatography (EtOAc 10%/DCM, then EtOAc 100%). The macrocyclic product 20 was isolated as a brown solid (200 mg). LC-MS (ESI, positive): 643 [M+H]⁺.

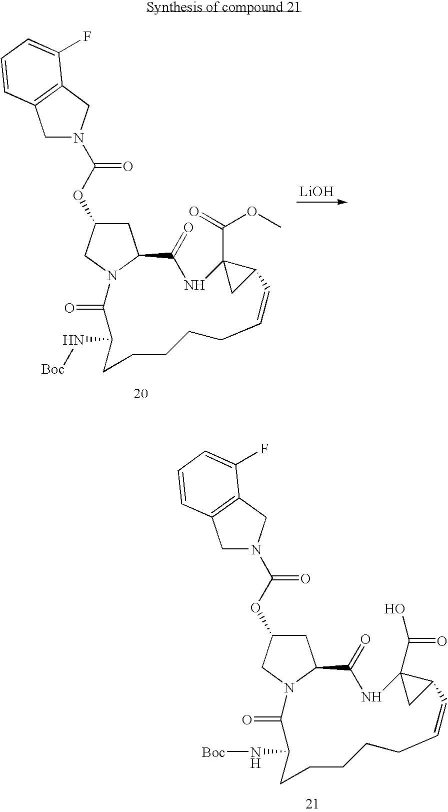

0.46 ml aqueous solution of LiOH (1N solution, 2 eq) was added to a solution of 20 (200 mg, 0.23 mmol) in 0.46 ml THF. The mixture was stirred at 30° C. for 2 h (completion of reaction confirmed by TLC). The reaction mixture was concentrated under reduced pressure, then diluted with DCM (50 mL), washed with HCl (1N) (20 mL) under 0° C., dried over anhydrous Na₂SO₄ and concentrated under reduced pressure to afford a brown solid. This crude product was purified by column chromatography (EtOAc100%, then methanol) to give compound 21 (100 mg). LC-MS (ESI, positive): 629 [M+H]⁺.

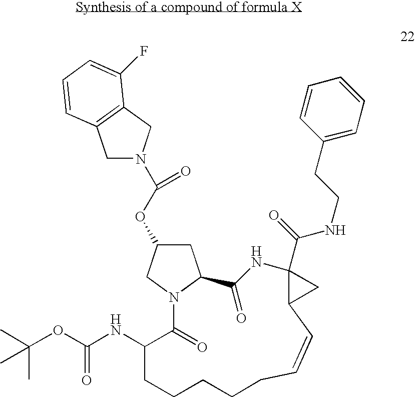

A solution of 21 (0.01 g, 0.016 mmol), HATU (0.007 g, 0.019 mmol), and DIEA (11.11 μL, 0.0636 mmol) in dry DMF was stirred for 1 h before the addition of a solution containing phenethylamine (0.003 g, 0.0239 mmol), DMAP (0.008 g, 0.0652 mmol), and DBU (9.8 μl, 0.0652 mmol) in dry DMF. The mixture was stirred at room temperature overnight. The solution was loaded onto a preparatory column (50-100% ACN) to obtain 4 mg of a solid compound 22, a compound of formula X. LC/MS 2.33 min, 732.33 (M+1; 100).

TABLE 1
Exemplary Structures of formula X

| EXAMPLES | |
|---|---|
| (prophetic | STRUCTURE |
| 2 | 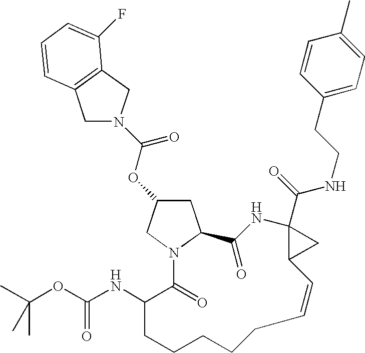 |
| 3 | 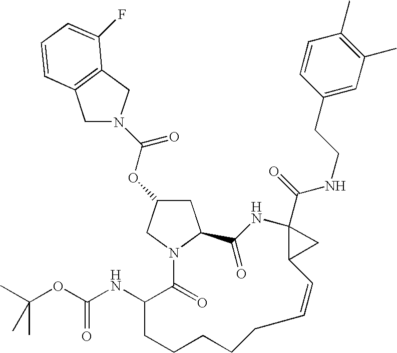 |
| 4 | 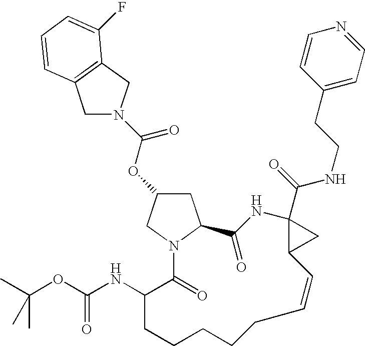 |
| 5 | 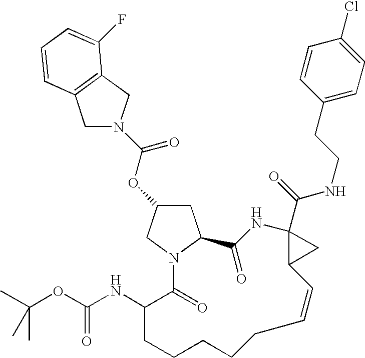 |
| 6 | 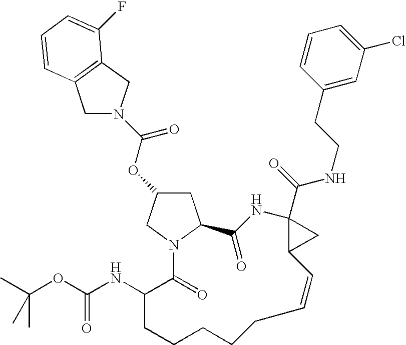 |
| 7 | 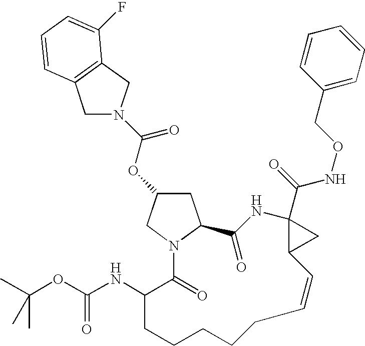 |
| 8 | 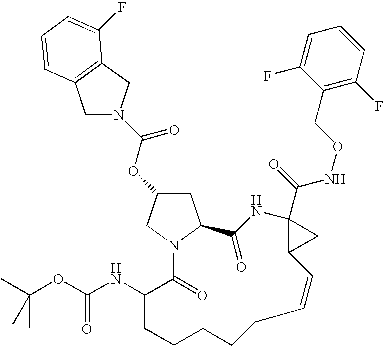 |
| 9 | 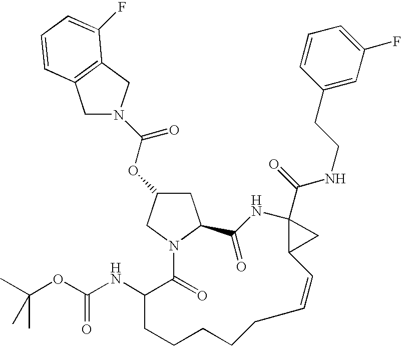 |
| 10 | 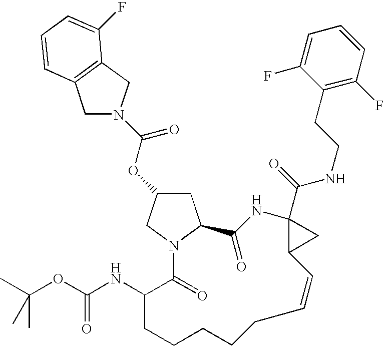 |
| 11 | 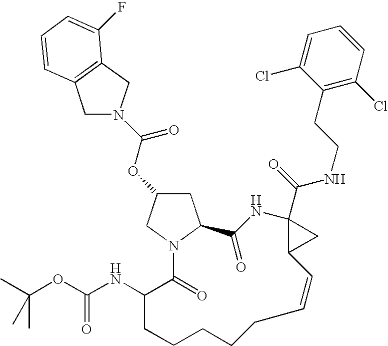 |
| 12 | 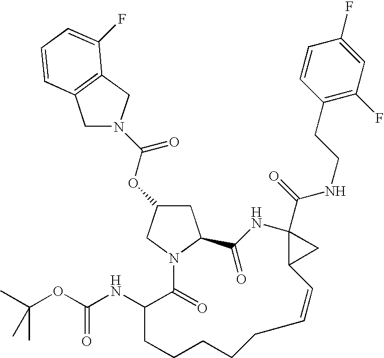 |

What is claimed is:

1. A compound of Formula X: 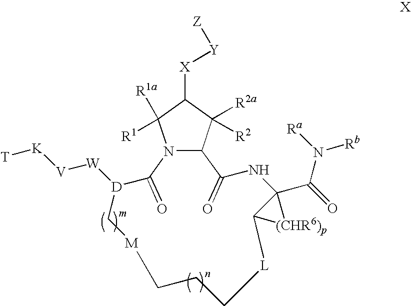
and stereoisomers, solvates, tautomers, prodrugs, salts, pharmaceutically acceptable salts, and mixtures thereof, wherein:
R^{a} and R^{b} at each occurrence are independently H, OR³, NR⁴R⁵, alkyl, alkenyl, aryl, aralkyl, aralkenyl, cycloalkyl, cycloalkylalkyl, cycloalkylalkenyl, cycloalkenyl, cycloalkenylalkyl, heterocyclyl, heterocyclylalkyl, heterocyclylalkenyl, heteroaryl, heteroarylalkyl, or heteroarylalkenyl, wherein any carbon atom can be substituted with J; or, R^{a} and R^{b}, together with a nitrogen atom to which they are bound, form a 3-8 membered heterocyclic ring which can be unsubstituted or substituted with 1-3 J, wherein the 3-8 membered heterocyclic ring can contain 1-3 additional heteroatoms selected from the group consisting of O, NR⁷, S, S(O), and S(O)₂, wherein the 3-8 membered heterocyclic ring can be fused with a substituted or unsubstituted, cycloalkyl, cycloalkenyl, aryl, heterocyclyl, or heteroaryl ring, or any combination thereof;
R¹, R^{1a}, R² and R^{2a} are independently H or alkyl, alkenyl, aryl, aralkyl, aralkenyl, cycloalkyl, cycloalkylalkyl, cycloalkylalkenyl, cycloalkenyl, cycloalkenylalkyl, heterocyclyl, heterocyclylalkyl, heterocyclylalkenyl, heteroaryl, heteroarylalkyl, or heteroarylalkenyl, wherein any carbon atom can be substituted with J;
R³, R⁴ and R⁵ are independently H or alkyl, alkenyl, aryl, aralkyl, aralkenyl, cycloalkyl, cycloalkylalkyl, cycloalkylalkenyl, cycloalkenyl, cycloalkenylalkyl, heterocyclyl, heterocyclylalkyl, heterocyclylalkenyl, heteroaryl, heteroarylalkyl, or heteroarylalkenyl, wherein any carbon atom can be substituted with J; or R⁴ and R⁵, together with a nitrogen atom to which they are bound, form a 3-8 membered heterocyclic ring which can be unsubstituted or substituted with 1-3 J, wherein the 3-8 membered heterocyclic ring can contain 1-3 additional heteroatoms selected from the group consisting of O, NR⁷, S, S(O), and S(O)₂, wherein the 3-8 membered heterocyclic ring can be fused with a cycloalkyl, cycloalkenyl, aryl, heterocyclyl, or heteroaryl ring, or any combination thereof;
D is CH₂, CH or N;
when D is CH₂, then W, V, K and T are absent;
when D is CH, then W is C(R⁶)₂, O, S, or NR⁷, and V, K, and T are as defined below;
when D is N then W, V and K are bonds, the bonds taken together forming a single bond, T is as defined below, such that T is bonded directly to D;
wherein R⁶ is independently at each occurrence hydrogen, alkyl, alkenyl, aryl, aralkyl, aralkenyl, cycloalkyl, cycloalkylalkyl, cycloalkylalkenyl, cycloalkenyl, cycloalkenylalkyl, heterocyclyl, heterocyclylalkyl, heterocyclylalkenyl, heteroaryl, heteroarylalkyl, or heteroarylalkenyl, wherein any carbon atom can be substituted with J; or wherein two R⁶ groups together with a carbon atom to which they are bond form a 3-8 membered cycloalkyl, which can be unsubstituted or substituted with 1-3 J, wherein the 3-8 membered cycloalkyl can contain 1-3 heteroatoms selected from the group consisting of O, NR⁷, S, S(O), and S(O)₂, wherein the 3-8 membered cycloalkyl can be fused with a cycloalkyl, cycloalkenyl, aryl, heterocyclyl, or heteroaryl ring, or any combination thereof;
R⁷ is independently at each occurrence hydrogen, alkyl, alkenyl, aryl, aralkyl, aralkenyl, cycloalkyl, cycloalkylalkyl, cycloalkylalkenyl, cycloalkenyl, cycloalkenylalkyl, heterocyclyl, heterocyclylalkyl, heterocyclylalkenyl, heteroaryl, heteroarylalkyl, or heteroarylalkenyl, wherein any carbon atom can be substituted with J, or aralkanoyl, heteroaralkanoyl, C(O)R⁸, SO₂R⁸ or carboxamido, wherein any aralkanoyl or heteroaralkanoyl is substituted with 0-3 J groups;
R⁸ is alkyl, alkenyl, aryl, aralkyl, aralkenyl, cycloalkyl, cycloalkylalkyl, cycloalkylalkenyl, cycloalkenyl, cycloalkenylalkyl, heterocyclyl, heterocyclylalkyl, heterocyclylalkenyl, heteroaryl, heteroarylalkyl, or heteroarylalkenyl, wherein any carbon atom can be substituted with J;
m is 1, 2, 3 or 4;
n is 0, 1, 2, 3 or 4;
p is 1, 2, 3, or 4;
M is O, S, S(O), S(O)₂, C(R⁶)₂ or N(R⁷);
J is halogen, R′, OR′, CN, CF₃, OCF₃, O, S, C(O), S(O), methylenedioxy, ethylenedioxy, (CH₂)₀₋₂N(R′)₂, (CH₂)₀₋₂SR′, (CH₂)₀₋₂S(O)R′, (CH₂)₀₋₂S(O)₂R′, (CH₂)₀₋₂S(O)₂N(R′)₂, (CH₂)₀₋₂SO₃R′, (CH₂)₀₋₂C(O)R′, (CH₂)ₒ₋₂C(O)C(O)R′, (CH₂)₀₋₂C(O)CH₂C(O)R′, (CH₂)₀₋₂C(S)R′, (CH₂)₀₋₂C(O)OR′, (CH₂)₀₋₂OC(O)R′, (CH₂)₀₋₂C(O)N(R′)₂, (CH₂)₀₋₂OC(O)N(R′)₂, (CH₂)₀₋₂C(S)N(R′)₂, (CH₂)₀₋₂NH—C(O)R′, (CH₂)₀₋₂N(R′)N(R′)C(O)R′, (CH₂)₀₋₂N(R′)N(R′)C(O)OR′, (CH₂)₀₋₂N(R)N(R′)CON(R′)₂, (CH₂)₀₋₂N(R′)SO₂R′, (CH₂)₀₋₂N(R′)SO₂N(R′)₂, (CH₂)₀₋₂N(R′)C(O)OR′, (CH₂)₀₋₂N(R′)C(O)R′, (CH₂)₀₋₂N(R′)C(S)R′, (CH₂)₀₋₂N(R′)C(O)N(R′)₂, (CH₂)₀₋₂N(R′)C(S)N(R′)₂, (CH₂)₀₋₂N(COR′)COR′, (CH₂)₀₋₂N(OR′)R′, (CH₂)₀₋₂C(═NH)N(R′)₂, (CH₂)₀₋₂C(O)N(OR′)R′, or (CH₂)₀₋₂C(═NOR′)R′; wherein,
each R′ is independently at each occurrence hydrogen, (C₁-C₁₂)-alkyl, (C₂-C₁₂)-alkenyl, (C₂-C₁₂)-alkynyl, (C₃-C₁₀)-cycloalkyl, (C₃-C₁₀)-cycloalkenyl, [(C₃-C₁₀)cycloalkyl or (C₃-C₁₀)-cycloalkenyl]-[(C₁-C₁₂)-alkyl or (C₂-C₁₂)-alkenyl or (C₂-C₁₂)-alkynyl], (C₆-C₁₀)-aryl, (Cₒ-C₁₀)-aryl-[(C₁-C₁₂)-alkyl or (C₂-C₁₂)-alkenyl or (C₂-C₁₂)-alkynyl], (C₃-C₁₀)-heterocyclyl, (C₃-C₁₀)-heterocyclyl-[(C₁-C₁₂)-alkyl or (C₂-C₁₂)-alkenyl or (C₂-C₁₂)-alkynyl], (C₅-C₁₀)-heteroaryl, or (C₅-C₁₀)-heteroaryl-[(C₁-C₁₂)-alkyl or (C₂-C₁₂)-alkenyl or (C₂-C₁₂)-alkynyl], wherein R′ is substituted with 0-3 substituents selected independently from J;
or, when two R′ are bound to a nitrogen atom or to two adjacent nitrogen atoms, the two R′ groups together with the nitrogen atom or atoms to which they are bound can form a 3- to 8-membered monocyclic heterocyclic ring, or an 8- to 20-membered, bicyclic or tricyclic, heterocyclic ring system, wherein any ring or ring system can further contain 1-3 additional heteroatoms selected from the group consisting of N, NR⁷, O, S, S(O) and S(O)₂, wherein each ring is substituted with 0-3 substituents selected independently from J;
wherein, in any bicyclic or tricyclic ring system, each ring is linearly fused, bridged, or spirocyclic, wherein each ring is either aromatic or nonaromatic, wherein each ring can be fused to a (C₆-C₁₀)aryl, (C₅-C₁₀) heteroaryl, (C₃-C₁₀)cycloalkyl or (C₃-C₁₀) heterocyclyl;
L is O, S, C₂, C₂H₂ or C₂H₄;
V is a bond,)C(R¹⁰)₂, C(O), S(O), or S(O)₂;
K is a bond, O, S, C(O), S(O), S(O)₂, S(O)(NR⁷), or N(R⁷);
provided that when V and K are both bonds, they form together a single bond;
R¹⁰ is independently at each occurrence hydrogen, alkyl, cycloalkyl, cycloalkylalkyl, aryl, aralkyl, heterocyclyl, heterocyclylalkyl, heteroaryl or heteroarylalkyl; or, two R¹⁰ groups together with a carbon atom to which they are bound form a 3-8 membered cycloalkyl, which can be unsubstituted or substituted with 1-3 J, wherein the 3-8 membered cycloalkyl can contain 1-3 heteroatoms selected from the group consisting of O, NR⁷, S, S(O), and S(O)₂, wherein the 3-8 membered cycloalkyl can be fused with a cycloalkyl, cycloalkenyl, aryl, heterocyclyl, or heteroaryl ring, or any combination thereof;
T is R¹¹, alkyl-R¹¹, alkenyl-R¹¹, alkynyl-R¹¹, OR¹¹, N(R¹¹)₂, C(O)R¹¹, or C(═NOalkyl)R¹¹;
R¹¹ is independently at each occurrence hydrogen, alkyl, aryl, aralkyl, alkoxy, amino, alkylamino, dialkylamino, cycloalkyl, cycloalkenyl, [cycloalkyl or cycloalkenyl]-[alkyl or alkenyl], heterocyclyl, heterocyclylalkyl, heterocyclylalkenyl, heteroaryl, heteroarylalkyl, or heteroarylalkenyl, wherein any R¹¹ except hydrogen is substituted with 0-3 J groups, or a first R¹¹ and a second R¹¹ together with a nitrogen atom to which they are bound form a mono- or bicyclic ring system substituted with 0-3 J groups and can contain 1-3 additional heteroatoms selected from the group consisting of O, NR⁷, S, S(O), and S(O)₂; and
when W is C(R⁶)₂, a bond, or absent;
X is a bond, O, S, CH(R⁶) or N(R⁷);
Y is a bond, CH(R⁶), C(O), C(O)C(O), S(O), S(O)₂, or S(O)(NR⁷);
provided that when both X and Y are bonds, they together form a single bond;
Z is:
a) hydrogen, alkyl, aryl, aralkyl, cycloalkyl, cycloalkylalkyl, heterocyclyl, heterocyclylalkyl, heteroaryl, heteroarylalkyl, OR⁹, or N(R⁹)₂, wherein any carbon atom is unsubstituted or is substituted with J, and wherein R⁹ is independently at each occurrence hydrogen, alkyl, alkenyl, aryl, aralkyl, aralkenyl, [cycloalkyl or cycloalkenyl]-[alkyl or alkenyl], heterocyclyl, heterocyclylalkyl, heterocyclylalkenyl, heteroaryl, or heteroarylalkyl, or two R⁹ groups together with a nitrogen atom to which they are bound can form together with the nitrogen atom a 5-11 membered mono- or bicyclic heterocyclic ring system substituted with 0-3 J groups and further including 0-3 additional heteroatoms selected from the group consisting of O, NR⁷, S, S(O), and S(O)₂;
or
b) a substituted aryl or heteroaryl group; wherein any aryl or heteroaryl is substituted with 1-3 J groups;
c) a group of the formula: 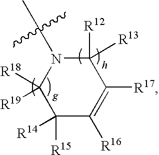
R¹², R¹³, R¹⁴, R¹⁵, R¹⁸ and R¹⁹ are independently hydrogen, fluorine, or a substituted or unsubstituted alkyl, cycloalkyl, cycloalkenyl, [cycloalkyl or cycloalkenyl]-[alkyl or alkenyl], aryl, aralkyl, aralkenyl, heterocyclyl, heterocyclylalkyl, heterocyclylalkenyl, heteroaryl, heteroarylalkyl, or heteroarylalkenyl group; or R¹² and R¹³ or R¹⁴ and R¹⁵ or R¹⁸ and R¹⁹, together with a carbon atom to which they are attached, form a C₃₋₆ cycloalkyl group;
R¹⁶ and R¹⁷ are independently hydrogen, fluorine, or a substituted or unsubstituted alkyl, cycloalkyl, cycloalkenyl, [cycloalkyl or cycloalkenyl]-[alkyl or alkenyl], aryl, aralkyl, aralkenyl, heterocyclyl, heterocyclylalkyl, heterocyclylalkenyl, heteroaryl, heteroarylalkyl, or heteroarylalkenyl group; or R¹⁶ and R¹⁷ together with the atoms to which they are attached form a fused substituted or unsubstituted aryl or heteroaryl group;
g is 0-1; and
h is 0-2;
or
d) a group of the formula: 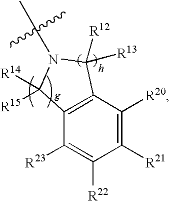
wherein
g is 0-2; and
h is 0-2;
R¹², R¹³, R¹⁴, and R¹⁵ are independently at each occurrence hydrogen, fluorine, or a substituted or unsubstituted alkyl, cycloalkyl, cycloalkenyl, [cycloalkyl or cycloalkenyl]-[alkyl or alkenyl], aryl, aralkyl, aralkenyl, heterocyclyl, heterocyclylalkyl, heterocyclylalkenyl, heteroaryl, heteroarylalkyl, or heteroarylalkenyl group; or R¹² and R¹³ or R¹⁴ and R¹⁵, together with a carbon atom to which they are attached, form a C₃₋₆ cycloalkyl group;
R²⁰, R²¹, R²², R²³ are independently H, F, Cl, Br, I, CN, CF₃, OCF₃, OR²⁴, (CH₂)_{q}OR²⁴, O(CH₂)_{q}OR²⁴, NR²⁵R²⁶, (CH₂)_{q}NR²⁵R²⁶, O(CH₂)_{q}NR²⁵R²⁶, SR²⁴, (CH₂)_{q}SR²⁴, O(CH₂)_{q}SR²⁴, C(O)R²⁴, (CH₂)_{q}C(O)R²⁴, O(CH₂)_{q}C(O)R²⁴, C(O)OR²⁴, (CH₂)_{q}C(O)OR²⁴, O(CH₂)_{q}C(O)OR²⁴, NR²⁷C(O)R²⁴, (CH₂)_{q}NR²⁷C(O)R²⁴, O(CH₂)_{q}NR²⁷C(O)R²⁴, C(O)NR²⁵R²⁶, (CH₂)_{q}C(O)NR²⁵R²⁶, O(CH₂)_{q}C(O)NR²⁵R²⁶, NR²⁷C(O)NR²⁵R²⁶, (CH₂)_{q}NR²⁷C(O)NR²⁶R²⁶, O(CH₂)_{q}NR²⁷C(O)NR²⁵R²⁶, OC(O)NR²⁵R²⁶, (CH₂)_{q}OC(O)NR²⁵R²⁶, O(CH₂)_{q}OC(O)NR²⁵R²⁶, NR²⁷C(O)OR²⁴, (CH₂)_{q}NR²⁷C(O)OR²⁴, O(CH₂)_{q}NR²⁷C(O)OR²⁴, NR²⁷SO₂R²⁴, (CH₂)_{q}NR²⁷SO₂R²⁴, O(CH₂)_{q}NR²⁷SO₂R²⁴, SO₂NR²⁵R²⁶, (CH₂)_{q}SO₂NR²⁵R²⁶, or O(CH₂)_{q}SO₂NR²⁵R²⁶, or a substituted or unsubstituted alkyl, cycloalkyl, cycloalkenyl, [cycloalkyl or cycloalkenyl]-[alkyl or alkenyl], aryl, aralkyl, aralkenyl, heterocyclyl, heterocyclylalkyl, heterocyclylalkenyl, heteroaryl, heteroarylalkyl, or heteroarylalkenyl group,
q is 1, 2, 3, 4, 5, or 6; and
each R²⁴, R²⁵, R²⁶, and R²⁷ is independently hydrogen, or a substituted or unsubstituted alkyl, cycloalkyl, cycloalkenyl, [cycloalkyl or cycloalkenyl]-[alkyl or alkenyl], aryl, aralkyl, arylalkenyl, heterocyclyl, heterocyclylalkyl, heterocyclylalkenyl, heteroaryl, heteroarylalkyl, or heteroarylalkenyl group; or R²⁵ and R²⁶ together with a nitrogen atom to which they are attached form a 3-7 membered heterocyclic ring substituted with 0-3 J groups, that further comprises 0-3 additional heteroatoms selected from the groups consisting of O, NR⁷, S, S(O), and S(O)₂;
or
e) a group of the formula 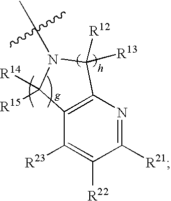
wherein
g is 0-2; and
h is 0-2;
R¹², R¹³, R¹⁴, R¹⁵, R²¹, R²² and R²³ are as defined in (d);
Or
f) a group of the formula 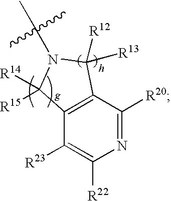
wherein
g is 0-2; and
h is 0-2;
R¹², R¹³, R¹⁴, R¹⁵, R²⁰, R²² and R²³ are as defined in (d); and
wherein a wavy line signifies a point of attachment;
and,
when W is NR⁷, O or S:
X is O, CH₂, or NR⁷;
Y is C(R⁶)₂ or absent;
Z is a substituted alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, alkoxy, aryloxy, alkylthio, arylthio, alkylamino, arylamino, heteroaryl, or heteroarylalkyl; wherein any alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, alkoxy, aryloxy, alkylthio, arylthio, alkylamino, arylamino, heteroaryl, or heteroarylalkyl is substituted with 1-3 J groups, provided that K and V are both bonds, taken together forming a single bond such that T is bonded directly to W, T is not C(O)R¹¹; or
X is O;
Y is C(O);
Z is
aa) a group of the formula 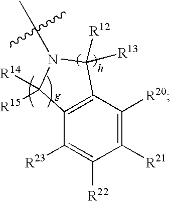
wherein
g is 0-2; and
h is 0-2;
R¹², R¹³, R¹⁴, and R¹⁵ are independently at each occurrence hydrogen, fluorine, or a substituted or unsubstituted alkyl, cycloalkyl, cycloalkenyl, [cycloalkyl or cycloalkenyl]-[alkyl or alkenyl], aryl, aralkyl, aralkenyl, heterocyclyl, heterocyclylalkyl, heterocyclylalkenyl, heteroaryl, heteroarylalkyl, or heteroarylalkenyl group; or R¹² and R¹³ or R¹⁴ and R¹⁵, together with a carbon atom to which they are attached, form a C₃₋₆ cycloalkyl group;
R²⁰, R²¹, R²², R²³ are independently H, F, Cl, Br, I, CN, CF₃, OCF₃, OR²⁴, (CH₂)_{q}OR²⁴, O(CH₂)_{q}OR²⁴, NR²⁵R²⁶, (CH₂)_{q}NR²⁵R²⁶, O(CH₂)_{q}NR²⁵R²⁶, SR²⁴, (CH₂)_{q}SR²⁴, O(CH₂)_{q}SR²⁴, C(O)R²⁴, (CH₂)_{q}C(O)R²⁴, O(CH₂)_{q}C(O)R²⁴, C(O)OR²⁴, (CH₂)_{q}C(O)OR²⁴, O(CH₂)_{q}C(O)OR²⁴, NR²⁷C(O)R²⁴, (CH₂)_{q}NR²⁷C(O)R²⁴, O(CH₂)_{q}NR²⁷C(O)R²⁴, C(O)NR²⁵R²⁶, (CH₂)_{q}C(O)NR²⁵R²⁶, O(CH₂)_{q}C(O)NR²⁵R²⁶, NR²⁷C(O)NR²⁵R²⁶, (CH₂)_{q}NR²⁷C(O)NR²⁵R²⁶, O(CH₂)_{q}NR²⁷C(O)NR²⁵R²⁶, OC(O)NR²⁵R²⁶, (CH₂)_{q}OC(O)NR²⁵R²⁶, O(CH₂)_{q}OC(O)NR²⁵R²⁶, NR²⁷C(O)OR²⁴, (CH₂)_{q}NR²⁷C(O)OR²⁴, O(CH₂)_{q}NR²⁷C(O)OR²⁴, NR²⁷SO₂R²⁴, (CH₂)_{q}NR²⁷SO₂R²⁴, O(CH₂)_{q}NR²⁷SO₂R²⁴, SO₂NR²⁵R²⁶, (CH₂)_{q}SO₂NR²⁵R²⁶, or O(CH₂)_{q}SO₂NR²⁵R²⁶, or a substituted or unsubstituted alkyl, cycloalkyl, cycloalkenyl, [cycloalkyl or cycloalkenyl]-[alkyl or alkenyl], aryl, aralkyl, aralkenyl, heterocyclyl, heterocyclylalkyl, heterocyclylalkenyl, heteroaryl, heteroarylalkyl, or heteroarylalkenyl group,
q is 1, 2, 3, 4, 5, or 6; and
each R²⁴, R²⁵, R²⁶, and R²⁷ is independently hydrogen, or a substituted or unsubstituted alkyl, cycloalkyl, cycloalkenyl, [cycloalkyl or cycloalkenyl]-[alkyl or alkenyl], aryl, aralkyl, arylalkenyl, heterocyclyl, heterocyclylalkyl, heterocyclylalkenyl, heteroaryl, heteroarylalkyl, or heteroarylalkenyl group; or R²⁵ and R²⁶ together with a nitrogen atom to which they are attached form a 3-7 membered heterocyclic ring substituted with 0-3 J groups, that further comprises 0-3 additional heteroatoms selected from the groups consisting of O, NR⁷, S, S(O), and S(O)₂;
or
bb) a group of the formula 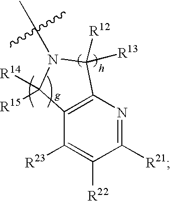
wherein
g is 0-2; and
h is 0-2;
R¹², R¹³, R¹⁴, R¹⁵, R²⁰, R²² and R²³ are as defined in (aa);
or
cc) a group of the formula 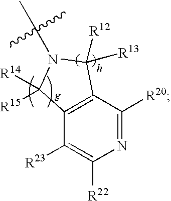
wherein
g is 0-2; and
h is 0-2;
R¹², R¹³, R¹⁴, R¹⁵, R²⁰, R²² and R₂₃ are as defined in (c);
wherein a wavy line signifies a point of attachment.

2. The compound of claim 1, wherein W is NR⁷.

3. The compound of claim 1 or 2, wherein X is O.

4. The compound of any one of claims 1-3, wherein Y is C(O).

5. The compound of any one of claims 1-4, wherein Z is a group of the formula:

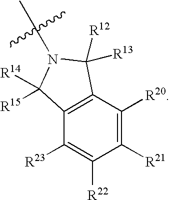

6. The compound of any claim 5, wherein R²⁰ is fluorine.

7. The compound of any one of claims 1-6, wherein Z is a group of the formula:

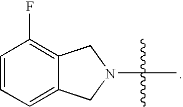

8. The compound of any one of claims 1-7, wherein R^{b} is H and R^{a} is 4-methylphenethyl, 3,4-dimethylphenethyl, 4-pyridylethyl, 4-chlorophenethyl, 3-chlorophenethyl, benzyloxy, 2,6-difluorobenzyloxy, 2,6-dichorophenyl, 3-fluorophenethyl, 2,6-difluorophenethyl, or 2,4-difluorophenethyl.

9. The compound of any one of claims 1-8, wherein V is C(O).

10. The compound of any one of claims 1-9, wherein K is O.

11. The compound of any one of claims 1-10, wherein R¹¹ is alkyl.

12. The compound of claim 11, wherein R¹¹ is tert-butyl.

13. The compound of any one of claims 1-12, wherein M is CH₂.

14. The compound of any one of claims 1-13, wherein L is C₂H₂, the C₂H₂ group being either in a Z or an E configuration.

15. The compound of claim 14, wherein M is CH₂, m is 1 and n is 1.

16. The compound of any one of claims 1-15, wherein p is 1.

17. The compound of any one of claims 1-16, comprising a compound of formula XI:

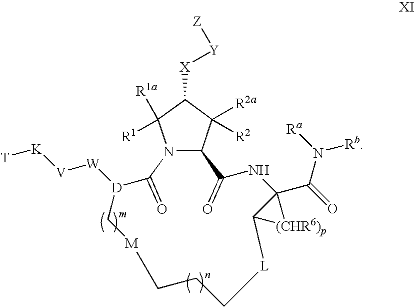

18. The compound of claim 1, comprising:

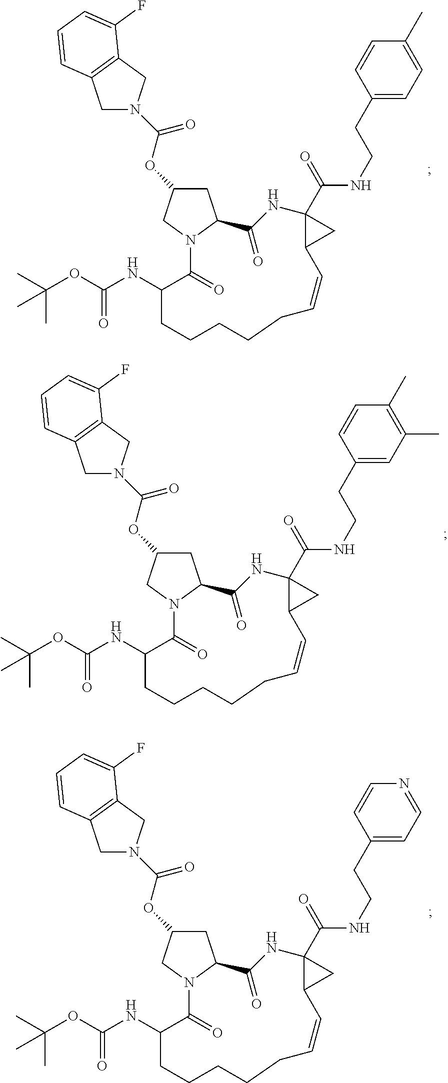

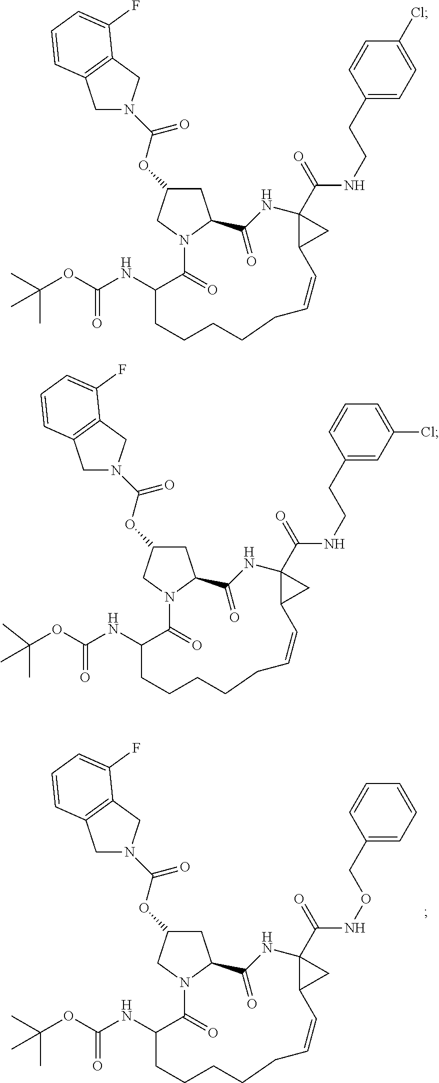

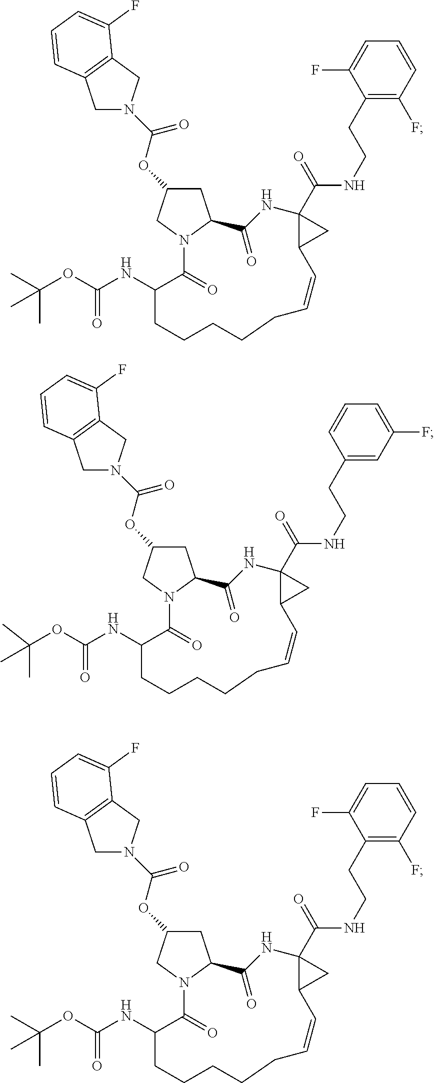

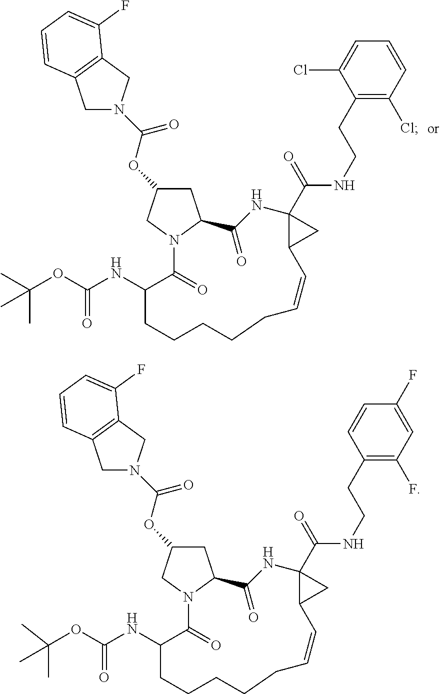

19. A pharmaceutical composition comprising a compound of any one of claims 1-18 and a suitable excipient.

20. A pharmaceutical combination comprising a compound of any one of claims 1-18 in a therapeutically effective dose and a second medicament in a therapeutically effective dose.

21. The pharmaceutical combination of claim 20 further comprising a third medicament in a therapeutically effective dose.

22. A pharmaceutical composition comprising the combination of claim 20 or 21 and a suitable excipient.

23. A method of treatment of a malcondition in a patient in need thereof, wherein inhibition of a hepatitis C viral protease is medically indicated, comprising administering to the patient a compound of any one of claims 1-18 or the composition of claim 19 in a therapeutically effective amount.

24. A method of treatment of a malcondition in a patient, the malcondition comprising a hepatitis C viral infection, comprising administering to the patient a compound of any one of claims 1-18 or the composition of claim 19 in a therapeutically effective amount.

25. A method of treatment of a malcondition in a patient, the malcondition comprising a hepatitis C viral infection or a condition where inhibition of a hepatitis C viral protease is medically indicated, comprising administering to the patient the pharmaceutical combination of claim 20 or 21 or the composition of claim 22 in a therapeutically effective amount.

26. The use of a compound of any one of claims 1-18 or the composition of claim 19 or 22, or the combination of claim 20 or 21, for the preparation of a medicament for use in treatment of any malcondition for which inhibition of a hepatitis C protease is medically indicated.

27. The use of a compound of any one of claims 1-18 or the composition of claim 19 or 22, or the combination of claim 20 or 21, for the preparation of a medicament for use in treatment of hepatitis C.

28. A method of preparation of a compound of claim 1, comprising contacting a compound of formula XII: 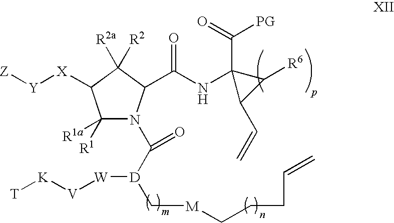
with a transition metal olefin metathesis catalyst in an amount, at a temperature, and for a duration effective to form the compound of formula XIII 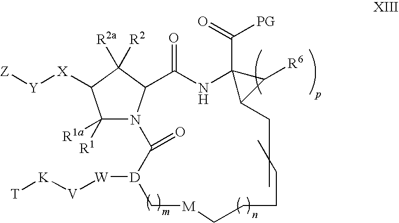
wherein PG is a carboxyl protecting group, then,
converting PG to NR^{a}R^{b} to provide a compound of formula X of claim 1 wherein L is C₂H₂.

29. The method of claim 28 wherein the transition metal olefin metathesis catalyst comprises benzylidene-bis(tricyclohexylphosphine)dichlororuthenium.
